SEQUENCE LISTING (41 sequences)

SEQ ID NO: 1 - protein, 5 aa, synthetic construct
RGDSP

SEQ ID NO: 2 - protein, 6 aa, synthetic construct
RGDSPK

SEQ ID NO: 3 - protein, 5 aa, synthetic construct
RGDTP

SEQ ID NO: 4 - protein, 10 aa, synthetic construct
RGDSPASSKP

SEQ ID NO: 5 - protein, 9 aa, synthetic construct
GGGGRGDSP

SEQ ID NO: 6 - protein, 6 aa, synthetic construct
PRRARV

SEQ ID NO: 7 - protein, 19 aa, synthetic construct
YEKPGSPPREWWPRPRPGW

SEQ ID NO: 8 - protein, 28 aa, synthetic construct
RPSLAKKORFRHRNRKGYRSORGHSRGR

SEQ ID NO: 9 - protein, 6 aa, synthetic construct
KQAGDV

SEQ ID NO: 10 - protein, 5 aa, synthetic construct
PHSRN

SEQ ID NO: 11 - protein, 5 aa, synthetic construct
YIGSR

SEQ ID NO: 12 - protein, 5 aa, synthetic construct
LRGDN

SEQ ID NO: 13 - protein, 5 aa, synthetic construct
PDSGR

SEQ ID NO: 14 - protein, 13 aa, synthetic construct
RLVSYNGIIFFLK

SEQ ID NO: 15 - protein, 4 aa, synthetic construct
DGEA

SEQ ID NO: 16 - protein, 4 aa, synthetic construct
RGDT

SEQ ID NO: 17 - protein, 4 aa, synthetic construct
VAPG

SEQ ID NO: 18 - protein, 4 aa, synthetic construct
REDV

SEQ ID NO: 19 - protein, 5 aa, synthetic construct
IKVAV

SEQ ID NO: 20 - protein, 6 aa, synthetic construct
GFOGER

SEQ ID NO: 21 - protein, 6 aa, synthetic construct
GLOGER

SEQ ID NO: 22 - protein, 12 aa, synthetic construct
DITWDQLWDLMK

SEQ ID NO: 23 - protein, 5 aa, synthetic construct
KDGEA

SEQ ID NO: 24 - protein, 14 aa, synthetic construct
GPAGGKDGEAGAQG

SEQ ID NO: 25 - protein, 7 aa, synthetic construct
FHRRIKA

SEQ ID NO: 26 - protein, 8 aa, synthetic construct
WQPPRARI

SEQ ID NO: 27 - protein, 13 aa, synthetic construct
RLVSYNGIIFFLK

SEQ ID NO: 28 - protein, 12 aa, synthetic construct
WKGWSYLWTQQA

SEQ ID NO: 29 - protein, 8 aa, synthetic construct
KPSSPPEE

SEQ ID NO: 30 - DNA, 22 bp, synthetic construct
  source 1..22: mol_type = other DNA
ctttgtcaagctcatttcctgg

SEQ ID NO: 31 - DNA, 19 bp, synthetic construct
  source 1..19: mol_type = other DNA
tcttgctcagtgtccttgc

SEQ ID NO: 32 - DNA, 22 bp, synthetic construct
  source 1..22: mol_type = other DNA
ctccaaaagctcaacaccaatg

SEQ ID NO: 33 - DNA, 19 bp, synthetic construct
  source 1..19: mol_type = other DNA
atttgtccatctccagccg

SEQ ID NO: 34 - DNA, 23 bp, synthetic construct
  source 1..23: mol_type = other DNA
gctattaaagtgacagtggacgg

SEQ ID NO: 35 - DNA, 22 bp, synthetic construct
  source 1..22: mol_type = other DNA
ggcgatcagagaacaaactagg

SEQ ID NO: 36 - DNA, 21 bp, synthetic construct
  source 1..21: mol_type = other DNA
tgttatgggtgaaactctggg

SEQ ID NO: 37 - DNA, 20 bp, synthetic construct
  source 1..20: mol_type = other DNA
agagctgattccgaagttgg

SEQ ID NO: 38 - DNA, 25 bp, synthetic construct
  source 1..25: mol_type = other DNA
catgtctcctgatatcctacacaac

SEQ ID NO: 39 - DNA, 21 bp, synthetic construct
  source 1..21: mol_type = other DNA
tgtcataatcctcttgccctg

SEQ ID NO: 40 - DNA, 20 bp, synthetic construct
  source 1..20: mol_type = other DNA
tgcttttcccttgtgagtcc

SEQ ID NO: 41 - DNA, 20 bp, synthetic construct
  source 1..20: mol_type = other DNA
tcagcttctcaatggtgtcc

(19) United States
(12) Patent Application Publication
(10) Pub. No.: US 2025/0179425 A1
(43) Pub. Date: Jun. 5, 2025

(54) Single Cell Encapsulation in Compartmentalized Hydrogel Coatings

(71) Applicant: THE BOARD OF TRUSTEES OF THE UNIVERSITY OF ILLINOIS, URBANA, IL (US)

(72) Inventors: JAE-WON SHIN, CHICAGO, IL (US); IK SUNG CHO, CHICAGO, IL (US)

(73) Assignee: THE BOARD OF TRUSTEES OF THE UNIVERSITY OF ILLINOIS, URBANA, IL (US)

(21) Appl. No.: 18/961,853

(22) Filed: Nov. 27, 2024

Related U.S. Application Data
(60) Provisional application No. 63/605,240, filed on Dec. 1, 2023.

(51) Int. Cl. C12N 5/00; A61L 27/38; C08B 37/00; C08J 3/24; C12M 3/06

(52) U.S. Cl. CPC C12N 5/0012; A61L 27/3834; C08B 37/0084; C08J 3/24; C12M 23/16

(57) ABSTRACT

Disclosed are single cells coated with a crosslinked alginate hydrogel layer comprising an asymmetrical distribution of cell surface receptor ligands within the alginate coating, as well as methods and devices for preparing the alginate-coated cells, compositions comprising the alginate-coated cells, and methods of using the alginate-coated cells to promote osteogenesis in a subject.

### CROSS-REFERENCE TO RELATED APPLICATIONS

This application claims benefit from U.S. Provisional Patent Application Ser. Nos. 63/605,240, filed Dec. 1, 2023, the contents of which are incorporated herein by reference in their entireties.

### FEDERALLY SPONSORED RESEARCH AND DEVELOPMENT

This invention was made with government support under grant nos. R01 GM141147 awarded by the National Institutes of Health and CAREER 2143857-CBET awarded by the National Science Foundation. The government has certain rights in this invention.

### STATEMENT REGARDING ELECTRONIC FILING OF A SEQUENCE LISTING

A Sequence Listing XML in format, entitled UIC0109US ST26.xml, 36,583 bytes in size, generated on Nov. 15, 2024, is filed herewith. This Sequence Listing is hereby incorporated herein by reference into the specification for its disclosures.

### BACKGROUND OF THE INVENTION

The extracellular matrix in tissues is composed of various biochemical, structural, and biophysical cues. At the microscale, cells often interact with local adhesive signals that are asymmetrically distributed. For example, bone marrow is composed of microscale domains with distinct matrix compositions, and some stem cells and progenitors in marrow are in contact with multiple domains in vivo. One important biological consequence of asymmetric cell-matrix interactions is the establishment of cell polarity. Defects in cell polarity have broad implications in driving pathological processes, including developmental defects, aging, and cancer. The process of establishing mammalian cell polarity has been studied on two-dimensional (2D) rigid cell culture substrates based on how protein complexes become spatially segregated to discrete regions of a cell over time. Spatial segregation of protein complexes changes cytoskeletal structures that dictate cell morphology and functions or alters the expression of regulatory factors that become partitioned into one of the daughter cells during division, leading to lineage specification.

To study cellular responses to asymmetric cell-matrix adhesion at the single cell resolution, it is important to spatially control the presentation of external cues at the microscale. Micropatterning was previously used to control the spatial presentation of matrix molecules underneath single cells and, hence, has served as a predominant approach to study cell polarity on 2D substrates. The anisotropic distribution of cell adhesion not only polarizes cytoskeleton organization but also drives cell migration and asymmetric segregation of DNA during cell division. In addition, micro-to-nanoscale variations in mechanics and topography of 2D substrates are known to result in polarized cell shape, directed migration, and even lineage specification. The maintenance of cell polarity upon adhesion on 2D substrates via integrins not only requires classical cell polarity regulators, such as the small GTPase Cdc42 but also involves the mechanotransduction machinery, including actomyosin contractility, nuclear lamins, and mechanosensitive transcription factors.

Cellular responses to asymmetric cell-matrix interactions in a three-dimensional (3D) space remain poorly understood and are inadequately controlled due to lack of methods to precisely tune material properties at the subcellular level in a 3D space. Studies have shown that cells can polarize and undergo spreading in degradable (Khetan et al. (2013) Nature Materials 12:458), fast stress relaxing (Adebowale et al. (2021) Nature Materials 20:1290) or plastic (Grolman et al. (2020) Proc. Natl. Acad. Sci. USA 117:25999) 3D hydrogels, where cells can overcome spatial confinement. However, cell polarity is likely established randomly in this context, since there is no directional signal present within hydrogels. Single cell encapsulation approaches are needed to enable the precision control of cell-matrix interactions at the single cell level. Encapsulating single cells in microwells fabricated within a bulk 3D hydrogel has shown the importance of niche geometry on stem cell shape, mechanotransduction, and differentiation (Bao et al. (2019) Nat. Commun. 8:1962; Bao et al. (2019) ACS Appl. Mater. Interfac. 11:1754). By using droplet-based microfluidics, it is possible to encapsulate single cells in microscale hydrogels (microgels) with lower material-to-cell volume ratios while tuning gel properties (Mao et al. (2017) Nat. Mater. 16:236; US 2023/0330147 A1), which could reveal biological phenotypes that were not previously observed with bulk hydrogels. For example, mesenchymal stem cells (MSCs) undergo adipogenic differentiation in an alginate-Arg-Gly-Asp (RGD) hydrogel with low elasticity (Huebsch et al. (2010) Nat. Mater. 9:518). However, when the amount of the alginate-RGD gel per cell is reduced while keeping the same gel elasticity, MSCs undergo osteogenic differentiation, which was shown to be associated with increased isotropic cell volume expansion and membrane tension in the presence of RGD (Wong et al. (2020) Adv. Sci. (Weinh) 7:2001066). However, existing single cell encapsulation approaches yield conformal gel coatings around single cells. Needed in the art is the ability to manipulate matrix-directed single cell polarity in a 3D space by compartmentalizing gel coatings where properties of each compartment can be independently controlled. While compartmentalized microgels have been fabricated to pattern different cell populations (Lu et al. (2015) J. Mater. Chem. B 3:353) or to pair cells (Zhang et al. (2018) Small 14(9)), single cell encapsulation in patterned gel coatings to control single cell polarity is needed. The present invention addresses this need in the art.

### SUMMARY OF THE INVENTION

Provided herein are alginate-coated cells comprising a crosslinked alginate hydrogel layer encapsulating single cells, wherein the alginate is conjugated to at least one cell surface receptor ligand, and wherein the at least one cell surface receptor ligand is unevenly distributed over the surface of each of the alginate-coated cells or distributed in a predefined location on the surface of each of the alginate-coated cells.

Also provided herein is a method of promoting osteogenesis in a subject in need of treatment comprising administering to the subject an effective amount of a composition comprising alginate-coated mesenchymal stem cells comprising a crosslinked alginate hydrogel layer encapsulating single mesenchymal stem cells, wherein the alginate is conjugated to at least one cell surface receptor ligand, and wherein the cell surface receptor ligand is unevenly distributed over the surface of each of the alginate-coated mesenchymal stem cells or distributed in predefined locations on the surface of each of the alginate-coated mesenchymal stem cells thereby promoting osteogenesis in the subject.

Further provided is a method of preparing a crosslinked alginate hydrogel layer encapsulating single cells, wherein the crosslinked alginate hydrogel is conjugated to at least one cell surface receptor ligand, and wherein the cell surface receptor ligand is unevenly distributed over the surface of each of the alginate-coated cells or distributed in predefined locations on the surface of each of the alginate-coated cells, comprising (i) providing a first aqueous phase comprising alginate conjugated to a first amount of a first cell surface receptor ligand; (ii) providing a second aqueous phase comprising unmodified alginate, cells, and a crosslinking agent; (iii) providing a third aqueous phase comprising unmodified alginate and alginate conjugated to a second amount of the first cell surface receptor ligand or alginate conjugated to a second cell surface receptor ligand; and (iv) simultaneously interfacing the first aqueous phase, second aqueous phase, and third aqueous phase with an oil phase that activates the crosslinking agent, thereby forming droplets comprising the crosslinked alginate hydrogel layer encapsulating single cells in which the first cell surface receptor ligand and optionally second cell surface receptor ligand is unevenly distributed over the surface of each of the alginate-coated cells or distributed in predefined locations on the surface of each of the alginate-coated cells.

A microfluidic device for preparing alginate-coated cells is also provided, which comprises a first microfluidic channel for conveying a first aqueous phase comprising alginate conjugated to a first amount of a first cell surface receptor ligand; a second microfluidic channel for conveying a second aqueous phase comprising unmodified alginate and cells coated with a crosslinking agent; a third microfluidic channel for conveying a third aqueous phase comprising unmodified alginate and alginate conjugated to a second amount of the first cell surface receptor ligand or alginate conjugated to a second cell surface receptor ligand; and a fourth microfluidic channel for conveying an oil phase comprising and an agent for activating the crosslinking agent; wherein the first, second, third and fourth microfluid channels interface at a junction whereby droplets comprising single cells encapsulated in a crosslinked alginate hydrogel layer are formed and exit the device via an outlet channel in fluid communication with the junction.

### BRIEF DESCRIPTION OF DRAWINGS

FIG. 1. Scheme illustrating the droplet microfluidic device to form conformal gel coatings with distinct compartments around single cells.

FIGS. 2A-2B. Gel crosslinking (X-link) is necessary upon emulsification to maintain cell viability in droplets. (FIG. 2A) Droplet size after collection. n=20 cells for each group. (FIG. 2B) Mean calcein intensity of single CaCO3-coated cells in droplets after collection in the presence or absence of acetic acid in the oil phase. N=20 cells. *p<0.0001 via Welch's T-test. The dotted horizontal line indicates a background mean intensity level. The device with height and width (in μm)=70 h, 70 w or 20 h, 30 w was used to form large and small gel coatings, respectively.

FIG. 3. Graph showing the volume of each gel compartment per gel-coated cell. Left: alginate-rhodamine, Right: alginate-CF™ 647. The device with height and width (in μm)=70 h, 70 w or 20 h, 30 w was used to form large and small gel coatings, respectively. n=18 for 70 h, 70 w, n=29 for 20 h, 30 w.

FIG. 4. Subcellular analysis of decay lifetime (τ) in compartmentalized microgels with asymmetric RGD presentation. Prior to encapsulation, MSCs were labelled with the membrane dye 5-hexadecanoylaminofluorescein (HEDAF), which serves as a donor. When HEDAF-labelled cells interact with the tetramethylrhodamine (TAMRA)-labelled integrin ligand Arg-Gly-Asp (T-RGD) conjugated to alginate microgels (˜60 μM at 1% w/v), T-RGD serves as an acceptor, leading to the reduction in donor fluorescence due to FRET. Fluorescence lifetime imaging microscopy (FLIM) was used to evaluate the decay lifetime (τ) of fluorescence. Shown are the τ values across different regions (angles in counterclockwise directions from 00) of the MSC cell membrane in a microgel composed of T-RGD in one compartment (T-RGD¹) and no RGD but with CF™ 350 in the other compartment (T-RGD⁰). n=10 cells. ***p<0.001. Individual p values were derived from Welch's ANOVA, followed by Dunnett T3 multiple comparisons test. All data are shown as mean±s.d.

FIGS. 5A-5D. Comparison of the kinetics parameters between symmetric (SYM) and asymmetric (ASYM) RGD presentations. The parameters include half-maximum times (t_{1/2}, in h) for (FIG. 5A) cytoplasmic and (FIG. 5B) nuclear volume expansion, and steady-state (s.s.) sphericity of (FIG. 5C) cells and (FIG. 5D) nuclei. n=3 independent experiments. *p<0.05, **p<0.01 via unpaired t-test. All data are shown as mean±s.d.

FIG. 6. Measurement of membrane tension at 1 day after encapsulation of MSCs in compartmentalized microgels. FLIM was used to evaluate the decay lifetime (τ) of the lipid tension reporter on the cell membrane. τ values across different regions (angles in counterclockwise directions from 00) of the MSC cell membrane in a microgel composed of ˜60 UM 1% w/v alginate-RGD (RGD¹) on the left side and unmodified RGD (RGD₀) on the right side are shown. n=10 cells. **p<0.01 via Welch's ANOVA, followed by Dunnett T3 multiple comparisons test. Data are shown as mean s.d.

FIGS. 7A-7B. Quantification of F-actin in F-tractin-tdTomato⁺ MSCs with symmetric (SYM) or asymmetric (ASYM) RGD presentation. (FIG. 7A) Ratio of F-tractin-tdTomato mean intensity between left and right compartments. (FIG. 7B) Ratio of tdTomato total intensity between the two compartments. n=18 cells. ***p <0.001 via Welch's T-test.

FIG. 8. Quantification of the ratio between the semi-major axis (aₓ, x:Left or Right) and the semi-minor axis of each cell in between two compartments. Images were taken by reconstructing a confocal z-stack, orienting the stack so that the boundary plane between the two compartments is vertical, and taking the midplane normal to the boundary plane. Shown is the quantification from n=15 cells per group. ns: not significant, *p<0.05, ****p<0.0001 via Welch's ANOVA, followed by Dunnett's T3 multiple comparisons test.

FIGS. 9A-9E. Polarized cell-matrix interactions drive osteogenic commitment of multipotent MSCs. After encapsulation in gels, MSCs were cultured for 1 day in the basal medium, followed by 10 days culture in presence of mixed osteogenic and adipogenic cocktails, and subsequent analyses. MSCs in compartmentalized microgels with symmetric (SYM) or asymmetric (ASYM) RGD distributions were compared with MSCs in the bulk alginate-RGD gel with the same elasticity (E˜2 kPa). Gene expression of osteogenic markers Alp (FIG. 9A) and Runx2 (FIG. 9B), and adipogenic marker Pparg1 (FIG. 9C). (FIG. 9D) Quantification of alkaline phosphatase (ALP) activity. (FIG. 9E) Quantification of lipid droplets by a fluorescent neutral lipid stain. n=3 independent experiments. n.s.: not significant, *p<0.05, **p<0.01, ***p<0.001, ****p<0.0001 via ordinary one-way ANOVA, followed by Tukey's multiple comparisons test. All data are shown as mean±s.d.

FIGS. 10A-10B. Subcellular localization of Rho GTPase activity with asymmetric cell-matrix interactions. Measurement of (FIG. 10A) Cdc42 activity and (FIG. 10B) Rac1 activity at 1 day after encapsulation of MSCs in compartmentalized microgels. Fluorescence lifetime imaging microscopy (FLIM) was used to quantify the decay lifetime (τ) of the donor (mTFP1-WASP CRIB for Cdc42, mTFP1-PBD for Rac1), which decreases upon FRET due to the interaction with Cdc42-Venus or Rac1-Venus. Shown are t values from MSCs in gels. n=10 cells. n.s.: not significant, **p<0.01 via Welch's ANOVA, followed by Dunnett T3 multiple comparisons test. Data are shown as mean±s.d.

### DETAILED DESCRIPTION OF THE INVENTION

A device and method for preparing a pure population of single cells encapsulated in a microscale layer of compartmentalized 3D hydrogel matrices has now been developed. Using the device and method, it is possible to precisely control the polarity of cell-matrix interactions by tuning spatial presentation of cell surface receptor ligands around single cells in a 3D space. Compartmentalized microgel coatings of single cells with asymmetric presentation of cell surface receptor ligands allow for single cells to become elongated in shape and undergo polarization of membrane tension. By comparison, cells expand isotropically when the cell surface receptor ligand is symmetrically presented at the surface of the cell. Membrane tension is higher on the side of single cells interacting with ligand than the side without ligand. Finite element analysis shows that a non-uniform isotropic cell volume expansion model is sufficient to recapitulate the process of single cell symmetry breaking in 3D matrices. This process is sufficient to commit single MSCs to the osteogenic lineage, demonstrating the functional implication of substrate-directed single stem cell polarization in lineage specification. In addition, modulation of small GTPases reveals Cdc42 as an essential mediator of symmetry breaking and lineage specification in response to polarized cell-matrix interactions. This study highlights the utility of precisely controlling 3D ligand presentation around single cells to direct cell polarity for tissue engineering and regenerative medicine.

Accordingly, provided herein are alginate-coated cells comprising a crosslinked alginate hydrogel layer encapsulating single cells, wherein the alginate is conjugated to at least one cell surface receptor ligand, and wherein the cell surface receptor ligand is unevenly distributed over the surface of each of the alginate-coated cells or distributed in predefined locations on the surface of each of the alginate-coated cells, and methods of preparing and using the same to promote osteogenesis.

As used herein, “alginate-coated cells” refers to individual or single cells coated with a biocompatible, biodegradable matrix composed of a crosslinked alginate hydrogel. In this respect, each individual cell is encapsulated by a single layer of crosslinked alginate hydrogel, e.g., in the form of a spherical core-shell microcapsule. This is distinct from a plurality of cells embedded in a layer of hydrogel matrix, i.e., bulk encapsulation. The hydrogel herein may be engineered to retain and release bioactive substances from cells in a spatially and temporally controlled manner. This controlled release not only eliminates systemic side effects and the need for multiple injections/infusions, but can be used to create a microenvironment that activates host cells at a hydrogel implant site and transplanted cells seeded onto/into a hydrogel.

“Biocompatible” generally refers to a material and any metabolites or degradation products thereof that are generally non-toxic to the recipient and do not cause any significant adverse effects to the subject. “Biodegradable” generally refers to a material that will degrade or erode by hydrolysis or enzymatic action under physiologic conditions to smaller units or chemical species that are capable of being metabolized, eliminated, or excreted by the subject. The degradation time is a function of polymer composition and morphology.

As used herein, a “hydrogel” refers to a substance formed when an organic polymer (natural or synthetic) is crosslinked via covalent, ionic, or hydrogen bonds to create a three-dimensional open-lattice structure which entraps water molecules to form a gel. “Biocompatible hydrogel” refers to a polymer which forms a gel which is not toxic to living cells, and allows sufficient diffusion of oxygen and nutrients to the entrapped cells to maintain viability.

“Alginate” is a collective term used to refer to linear polysaccharides formed from (1-4)-linked β-D-mannuronic acid monomers (M units) and L-guluronic acid monomers (G units) in any M/G ratio and sequential distribution along the polymer chain, as well as salts and derivatives thereof. In certain aspects, an alginate of use in the preparation of the hydrogel herein may have a molecular weight of at least about 250 kDa (e.g., about 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 410 kDa, 420 kDa, 430 kDa, 440 kDa, 450 kDa, 460 kDa, 470 kDa, 480 kDa, 490 kDa, or 500 kDa, or any value or range therebetween). In some aspects, an alginate of use in the preparation of the hydrogel herein may have a molecular weight in the range of about 250 kDa to about 500 kDa.

As used herein, the term “about” means that the amount or value can be the specific value designated or some other value nearby. In general, the term “about” used to indicate a specific value is intended to indicate a range within +5% of that value. As an example, the phrase “about 100” refers to the range of 100±5, i.e., from 95 to 105. In general, when the term “about” is used, it can be expected that similar results or effects according to the present invention can be obtained within +5% of the specified value.

In some aspects, alginate monomers may be crosslinked via ionic bonds to form the hydrogel layer encapsulating the individual cells. Unlike many other gelation procedures, ionic crosslinking can occur at pH, temperature and salt conditions that maintain cell viability and/or protein activity. Ionic crosslinking of alginate monomers may be carried out using one or more divalent or trivalent cations. In some aspects, the crosslinked alginate hydrogel herein may be prepared by crosslinking (1-4)-linked β-D-mannuronic acid and L-guluronic acid monomers with one or more of Ca²⁺, Mg²⁺, Sr²⁺, Ba²⁺, Be²⁺, Al³⁺, or a combination thereof. In some aspects, alginate monomers may be crosslinked with a divalent cation. In some aspects, alginate monomers may be crosslinked with Ca²⁺, Sr²⁺, Ba²⁺, or a combination thereof. In other aspects, the alginate monomers may be crosslinked with Ca²⁺.

Alternatively, alginate monomers may be crosslinked via covalent bonds. Carboxylate groups present on the monomer chains provide a region for relatively straight forward modification. For example, alginate may be modified by functionalizing carboxylate moieties with methacrylates such as 2-aminoethyl methacrylate hydrochloride, which allows for photo crosslinking (Somo, et al. (2018) Acta Biomater. 65:53; Chou, et al. (2009) Osteoarthritis Cartilage 17(10): 1377-84; Samorezov, et al. (2015) Bioconjug. Chem. 26(7): 1339-47). One or a combination of ionic and covalent crosslinking may be used to modify the mechanical stability, stiffness, and/or utility of the hydrogel herein.

As used herein, the term “layer” refers to a coating on the surface of a cell, e.g., a shell formed around the cell, wherein the coating has a defined thickness. In some aspects, single or individual cells may be coated with a crosslinked alginate hydrogel layer having a thickness of up to about 30 microns. In some aspects, the thickness of the crosslinked alginate layer may be less than about 30 microns (e.g., less than about 29, 28, 27, 26, 25, 24, 23, 22, 21, 20, 19, 18, 17, 16, 15, 14, 13, 12, 10, 9, 8, 7, 6, 5, 4 microns, or less than about 3 microns, or any value or range therebetween). In some aspects, a crosslinked alginate hydrogel coating of an individual or single cell may have a thickness of about 0.5 to about 30 microns (e.g., about 0.5 to about 29 microns, 0.5 to about 25 microns, 0.5 to about 20 microns, 0.5 to about 10 microns, 0.5 to about 5 microns, 1 to about 2 microns, 1 to about 3 microns, 1 to about 4 microns, 1 to about 5 microns, 2 to about 3 microns, 2 to about 4 microns, 2 to about 5 microns, 3 to about 4 microns, 3 to about 5 microns, 4 to about 5 microns, 5 to about 10 microns, 55 to about 15 microns, 5 to about 20 microns, 5 to about 25 microns, 5 to about 30 microns, 10 to about 15 microns, 10 to about 20 microns, 10 to about 25 microns, 10 to about 30 microns, 15 to about 20 microns, about 15 to about 20 microns, about 15 to about 25 microns, or about 15 to about 30 microns).

In some aspects, a crosslinked alginate hydrogel layer encapsulating single cells may be further characterized by low stiffness, e.g., have Young's modulus of about 0.1 to about 10 kPa. In some aspects, the hydrogel may have a Young's modulus of 0.1 to about 10 kPa (e.g., about 0.1 to about 9 kPa, 0.1 to about 8 kPa, 0.1 to about 7 kPa, 0.1 to about 6 kPa, 0.1 to about 5 kPa, 0.1 to about 4 kPa, 0.1 to about 3 kPa, 0.1 to about 2 kPa, 0.1 to about 1 kPa, 0.5 to about 1 kPa, 0.5 to about 2 kPa, 0.5 to about 3 kPa, 0.5 to about 4 kPa, or about 0.5 to about 5 kPa, or any value or range therebetween). In some aspects, the crosslinked alginate hydrogel encapsulating the single cells may have a Young's modulus of about 2 kPa.

In some aspects, an alginate hydrogel coating may be characterized by a stress relaxation rate (τ_{1/2}) of 10 seconds or less (e.g., 9 seconds or less, 8 seconds or less, 7 seconds or less, 6 seconds or less, 5 seconds or less, e.g., 4 seconds, 3 seconds, 2 seconds, 1 second, 0.5 second, 0.1 second or less). In some aspects, the alginate hydrogel coating may be characterized by a stress relaxation rate in the range of about 0.1 second to about 10 seconds.

In some aspects, the diameter of the encapsulated cells may be in the range of about 7 to about 30 microns (e.g., about 7, 8, 9, 10, 11, 12, 13, 14, 15, 16, 17, 18, 19, 20, 21, 22, 23, 24, 25, 26, 27, 28, 29 or 30 microns, or any range therebetween). In some aspects the diameter of the encapsulated cells may be in the range of about 15 to 20 microns. The volume or size of encapsulated single cells may depend on both the cell adhesion molecule and hydrogel coating thickness. For example, when cells are in thinner hydrogels (i.e., a crosslinked alginate hydrogel layer having a thickness of about 5 microns) that are functionalized with a cell adhesive ligand, they may expand by 50% more rapidly than when cells are in thicker hydrogels (i.e., a crosslinked alginate hydrogel layer having a thickness of about 15 microns).

According the compositions and methods herein, the crosslinked alginate hydrogel is conjugated to one or more cell surface receptor ligands. A “cell surface receptor ligand,” “receptor ligand,” “cell receptor ligand” or “ligand” is used herein to refer a molecule that facilitates attachment of the alginate to the cell surface by being recognized by and binding to a cell surface receptor. A cell surface receptor ligands may be a polysaccharide and/or peptide/protein. Examples of suitable polysaccharide-based ligands include hyaluronic acid or chondroitin. Suitable peptide- or protein-based ligands may be derived from derived or obtained from fibronectin, vitronectin, laminin, collagen, elastin, or thrombospondin. Exemplary peptide-based ligands may include those having the sequence RGD (e.g., RGDSP (SEQ ID NO:1), RGDSPK (SEQ ID NO: 2), RGDTP (SEQ ID NO: 3), RGDSPASSKP (SEQ ID NO: 4), or GGGGRGDSP (SEQ ID NO: 5)), PRRARV (SEQ ID NO: 6), or YEKPGSPPREWWPRPRPGW (SEQ ID NO: 7) derived from fibronectin; RPSLAKKORFRHRNRKGYRSORGHSRGR (SEQ ID NO: 8) derived from vitronectin; KQAGDV (SEQ ID NO: 9), PHSRN (SEQ ID NO: 10), YIGSR (SEQ ID NO:11), LRGDN (SEQ ID NO: 12), PDSGR (SEQ ID NO:13), or RLVSYNGIIFFLK (SEQ ID NO:14) derived from laminin; DGEA (SEQ ID NO: 15) or RGDT (SEQ ID NO: 16) derived from collagen; VTXG derived from thrombospondin; or VAPG (SEQ ID NO: 17) derived from elastin. Other examples of peptide-based ligands include, but are not limited to, peptides have the amino acid sequence REDV (SEQ ID NO: 18), IKVAV (SEQ ID NO: 19), GFOGER (SEQ ID NO: 20), GLOGER (SEQ ID NO: 21), DITWDQLWDLMK (SEQ ID NO: 22), KDGEA (SEQ ID NO:23), GPAGGKDGEAGAQG (SEQ ID NO: 24), FHRRIKA (SEQ ID NO: 25, derived from a bone sialoprotein), WOPPRARI (SEQ ID NO: 26), and CD44 ligands RLVSYNGIIFFLK (SEQ ID NO: 27), WKGWSYLWTQQA (SEQ ID NO: 28), and KPSSPPEE (SEQ ID NO: 29). In some aspects, one or more cell surface receptor ligands attached to alginate chains may be synthetic peptides containing RGD amino acid sequences found in various natural extracellular matrix molecules.

As used herein, the following abbreviations are used in describing amino acids, peptides, or proteins: Ala or A, Alanine; Arg or R, Arginine; Asn or N asparagine, Asp or D, aspartic acid; Cys or C, cysteine; Gln or Q, glutamine; Glu or E, glutamic acid; Gly or G, glycine; His or H, histidine; Ile or I, isoleucine; Leu or L, leucine; Lys or K, lysine; Met or M, methionine; Phe or F, phenylalanine; Pro or P, proline; Ser or S, serine; Thr or T, threonine; Trp or W, tryptophan; Tyr or Y, tyrosine; and Val or V, valine.

Covalent conjugation or coupling of cell surface receptor ligands to alginate may be performed using synthetic techniques commonly known to those skilled in the art and exemplified herein. A particularly useful method is by forming an amide bond between the carboxylic acid group on the alginate chain and the amino group on the cell adhesion molecule. Other useful adhesion chemistries include those discussed by Hermanson ((1996) Bioconjugate Techniques, p. 152-183).

In some aspects, the one or more cell surface receptor ligands may be unevenly distributed over the surface of each of the alginate-coated cells, i.e., the alginate coating unevenly or asymmetrically presents or provides one or more cell surface receptor ligands to the surface of each of the cells. As used herein, “unevenly distributed” refers to a phenomenon in which the concentration or amount of one or more cell surface receptor ligands is not uniform or homogenous over the entire surface of an alginate-coated cell. In some aspects, “unevenly distributed” refers to a phenomenon in which the concentration or amount of a cell surface receptor ligand may be high in one location on the surface of a cell, but low (e.g., 2-fold, 3-fold, 4-fold, 5-fold, 10-fold, or as much as 100-fold lower) at another location on the surface of the same cell.

In some aspects, the one or more cell surface receptor ligands may be distributed in predefined locations on the surface of each of the alginate-coated cells, i.e., the alginate coating presents or provides one or more cell surface receptor ligands to predefined locations on the surface of each of the cells. By “predefined locations” on the surface of a cell it is meant that the position of the one or more cell surface receptor ligands is fixed relative to the surface of a cell. In some aspects, a cells may be divided in half, wherein one half (e.g., a first predefined location) has a first concentration or amount of one or more cell surface receptor ligands and the other half (e.g., a second predefined location) has a second concentration or amount of one or more cell surface receptor ligands.

In some aspects, a concentration or amount of a cell surface receptor ligand may be at least about 1.5-fold or more (e.g., 1.5-, 1.6-, 1.7-, 1.8-, 1.9-, 2.0-, 2.5-, 3.0-, 3.5-, 4.0-, 4.5-, 5.0-, 5.5-, 6.0-, 6.5-, 7.0-, 7.5-, 8.0-, 8.5-, 9.0-, 9.5-, 10.0-fold or more) at one location (e.g., a first predefined location) on the surface of an alginate-coated cell as compared to the concentration or amount of the same cell surface receptor ligand at another location (e.g., a second predefined location) on the surface of an alginate-coated cell. In some aspects, a concentration or amount of a cell surface receptor ligand at a first predefined location and the concentration or amount of the cell surface receptor ligand at a second predefined location may be at a ratio greater than 1:1, e.g., at least 1:1.25, 1:1.5, 1:75, 1:2, 1:3, 1:4, 1:5, 1:6, 1:7, 1:8, 1:9, 1:10 up to 1:100, or any value or range therebetween.

In some aspects, the uneven distribution of a cell surface receptor ligand or distribution of a cell surface receptor ligand in predefined locations on the surface of each of the alginate-coated cells results in the elongation of the alginate-coated cells as compared to alginate-coated cells where the cell surface receptor ligand is evenly or homogenously distributed over the surface of the cells. As used herein, the term “elongation” or “elongated” means that the cell is not substantially spherical and takes on an oval shape.

In some aspects, cells of use in the compositions and methods described herein capable of undergoing cell polarization. “Cell polarization” refers to a self-organizing process that breaks a cell's internal symmetry by establishing a preferred axis. This process is important for many cellular functions, including cell migration, differentiation, localized membrane growth, activation of the immune response, and vectorial transport of molecules across cell layers. In some aspects, cells capable of undergoing cell polarization include, but are not limited to, hematopoietic cells, immune cells, epithelial cells, endothelial cells, stromal cells, and mesenchymal cells.

In some aspects, cells capable of undergoing cell polarization are mesenchymal stem cells. As used herein, “mesenchymal stromal cells” (also referred to as “mesenchymal stem cell” or “MSCs”) are multipotent cells that can differentiate into a variety of progenitor cell types including connective tissue, bone, cartilage, and cells in the circulatory and lymphatic systems. Mesenchymal stromal cells may be found in the mesenchyme, the part of the embryonic mesoderm that consists of loosely packed, fusiform or stellate unspecialized cells. Mesenchymal stromal cells may be obtained by conventional methods and may be identified one or more of the following markers: CD29⁺, CD31⁻, CD34⁻, CD44⁺CD45⁻, CD51⁺, CD73⁺, CD90/Thy-1⁺, CD105⁺, CD166⁺, Integrin alt, PDGF Rα⁺, Nestin⁺, Sca-1⁺, SCF R/c-Kit⁺, STRO-1⁺, and/or VCAM-1⁺. In some aspects, the mesenchymal stromal cells may be derived or obtained from bone marrow (BM), skeletal muscle, lung tissue, cord blood, adipose tissue (ASC) and the like. In some aspects, the MSCs may be obtained from the same tissue in which the alginate-coated cells are intended to subsequently be used to treat. For example, bone marrow-derived MSCs may be isolated, coated with alginate, and used for bone repair; skeletal muscle-derived MSCs may be isolated, coated with alginate, and used for the treatment of muscle fibrosis; and lung-derived MSCs may be isolated, coated with alginate, and used for the treatment of lung fibrosis. In some aspects, mesenchymal stromal cells may be derived or obtained from human bone marrow.

The cells used in the compositions and methods herein may be autologous or heterologous, e.g., allogeneic. “Autologous” refers to a transplanted biological substance taken from the same individual. “Allogeneic” refers to a transplanted biological substance taken from a different individual of the same species.

The cells used in the preparation of the alginate-coated cells herein may be isolated and optionally purified. As used herein the term “isolated” is meant to describe a cell of interest that is in an environment different from that in which the element naturally occurs. “Purified” as used herein refers to a cell removed from an environment in which it was produced and is at least 60% free, preferably 75% free, and most preferably 90% free from other components with which it is naturally associated or with which it was otherwise associated with during production.

Purification and/or identification of cells of interest can be achieved through any means known in the art, for example, immunologically. Histochemical staining, flow cytometry, fluorescence-activated cell sorting (FACS), western blot analysis, enzyme-linked immunosorbent assay (ELISA), and the like may be used. Flow immunocytochemistry may be used to detect cell-surface markers and immunohistochemistry (for example, of fixed cells) may be used for intracellular or cell-surface markers. Western blot analysis may be conducted on cellular extracts. Enzyme-linked immunosorbent assay may be used for cellular extracts or products secreted into the medium. Antibodies for the identification of stem cell markers may be obtained from commercial sources, for example from Biolegend (San Diego, CA).

Depending on the intended use of the alginate-coated cells, the crosslinked alginate hydrogel layer may further include one or more active ingredients such as growth h factors, inflammatory factors, and/or differentiation factors. Such active ingredients may be included during the preparation of the alginate hydrogel layer or after encapsulation of the cells in the crosslinked alginate hydrogel layer. In the latter case, the active ingredient may be provided to the encapsulated cells and diffuse through the hydrogel gel.

In some aspects, crosslinked alginate hydrogel encapsulating single cells (e.g., single MSCs) may further comprise one or more growth factors. As used herein, a “growth factor” is a substance, which stimulates the growth of living cells. Examples of suitable growth factors that may be included or embedded in the crosslinked alginate hydrogel layer are Bmp1, Bmp2, Bmp3, Bmp4, Bmp5, Bmp6, Bmp7, Bmp8A, Bmp8B, Clec11a, Ostn, Chrdl1, Co11a1, Colla2, Col5a1, Col5a2, Col5a3, Col6a1, Col6a2, Col6a3, Col13a1, Ecm1, Pkdcc, Fn1, Fst13, Gdf2, Gdf3, Gdf10, Igsf10, Ifitm1, Kazald1, LTF, Lrrc17, Mgp, Lamb3, TGFB1, TGFB3, PDGF, VEGF, PTH, IGF1, FGF2, FGF9, BGLAP2, BGLAP3, PRG2, MEPE, and the like, as well as agonistic peptides of the same. Agonists of receptors of the above referenced growth factors may also be used. In some aspects, the growth factor is Bmp2, a Bmp2-derived agonist peptide, or a BMP receptor agonist.

In some aspect, a composition comprising the alginate-coated cells described herein and a pharmaceutically acceptable carrier or aqueous medium is also provided. Compositions containing the alginate-coated cells may be prepared by combining the alginate-cells with a pharmaceutically acceptable carrier or aqueous medium. The phrase “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” may include any and all solvents, dispersion media, coatings, antibacterial and antifungal agents, isotonic and the like. The use of such media and agents for pharmaceutically active substances is well known in the art. Except insofar as any conventional media or agent is incompatible with the cells herein, its use in therapeutic compositions is contemplated. Appropriate compositions may be prepared by one skilled in the art depending upon, for example, the intended route of administration, delivery format and desired dosage. See, for example, Remington, J. P. & Allen, L. V. (2013) Remington: The Science and Practice of Pharmacy. London, Pharmaceutical Press.

Compositions containing alginate-coated cells may be incorporated in an injectable formulation. The formulation may also include the necessary physiologically acceptable carrier material, excipient, lubricant, buffer, surfactant, antibacterial, bulking agent (such as mannitol), antioxidants (ascorbic acid or sodium bisulfite) and the like.

Acceptable formulation materials are generally nontoxic to recipients at the dosages and concentrations employed. A composition herein may contain formulation materials for modifying, maintaining or preserving, for example, the pH, osmolarity, clarity, viscosity, color, isotonicity, odor, sterility, stability, rate of dissolution or release, adsorption or penetration of the composition. Suitable formulation materials may include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA; complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as poloxamers, PEG, sorbitan esters, polysorbates such as polysorbate 20 and polysorbate 80, Triton™, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington, J. P. & Allen, L. V. (2013) Remington: The Science and Practice of Pharmacy. London, Pharmaceutical Press.

The primary vehicle or carrier in a pharmaceutical composition may be either aqueous or nonaqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions may include Tris buffer of about pH 7.0-8.5, or acetate buffer of about pH 4.0-5.5, which may further include sorbitol a or suitable substitute therefore. Pharmaceutical compositions herein may be prepared for storage by mixing the selected composition having the desired degree of purity with optional formulation agents (Remington, J. P. & Allen, L. V. (2013) Remington: The Science and Practice of Pharmacy. London, Pharmaceutical Press) in the form of a lyophilized cake or an aqueous solution.

The alginate-coated cells or composition containing the same may be provided by sustained release systems, by encapsulation or by implantation devices. A composition herein may be administered by bolus injection or continuously by infusion, or by implantation device. A composition herein may also be administered locally via implantation of a membrane, sponge or another appropriate material onto which the alginate-coated cells have been absorbed or encapsulated. Where an implantation device is used, the device may be implanted into any suitable tissue or organ. The injections may be given as a one-time treatment, repeated (daily, weekly, monthly, annually, etc.) to achieve the desired therapeutic effect.

A composition herein be delivered parenterally. When parenteral administration is contemplated, the compositions may be in the form of a pyrogen-free, parenterally acceptable aqueous solution. A particularly suitable vehicle for parenteral injection is sterile distilled water. Preparation may involve the formulation with an agent, such as injectable microspheres, bio-erodible particles, polymeric compounds (such as polylactic acid or polyglycolic acid), beads or liposomes, that may provide controlled or sustained release of the alginate-coated cells, which may then be delivered via a depot injection. Formulation with hyaluronic acid has the effect of promoting sustained duration in the circulation. Implantable drug delivery devices may be used to introduce the desired composition.

Compositions containing alginate-coated cells may also contain adjuvants such as preservative, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents such as sugars, sodium chloride and the like.

In addition to those described above, other pharmaceutical preparations are also contemplated. Administration of these compositions may be via any common route so long as the target tissue is available via that route. Such routes include oral, nasal, buccal, rectal, vaginal or topical route. Ideally, administration may be by intratracheal instillation, intratracheal inhalation, intravenous delivery, intramuscular delivery, intraarterial delivery, topical delivery, renal artery injection, portal vein injection, intrabone delivery, intraarticular delivery, intralymphatic delivery, intrathymic delivery, intrarenal delivery, intracorneal delivery, intraportal delivery, intrahepatic delivery, or intracardiac injection of the alginate-coated cells. Such compositions may be administered as pharmaceutically acceptable compositions.

In some aspects, a composition containing alginate-coated cells may further include one or more ion channel modulators, cell contractility modulators, small GTPase modulators, or a combination thereof. In some aspects, an exemplary ion channel modulator includes, but is not limited to, GSK1016790A (a TRPV4 agonist; CAS No. 942206-85-1). In some aspects, suitable cell contractility modulators include, but are not limited to, 4′-hydroxyacetophenone (a myosin-II activator; CAS No. 99-93-4) or EMD57033 (an actomyosin ATPase agonist; CAS No. 147527-31-9). In some aspects, an exemplary small GTPase modulator includes, but is not limited to, Rho/Rac/Cdc42 Activator I CN04 (commercially available from Cytoskeleton, Inc., Denver, CO.

As demonstrated herein, the alginate-coated cells of this invention are of particular use in cell-based therapies. In some aspects, the alginate-coated cells and compositions containing the same may be of use in the treatment of diseases or conditions in which a subject may receive benefit from a polarized cell, e.g., bone regrowth and repair, subject with defects in cell migration or differentiation, subjects with immune response defects or deficiencies, and subjects with neuronal outgrowth defects or deficiencies. In one aspect, a method of promoting osteogenesis in a subject is provided. In accordance with this method, a subject in need of treatment is administered (e.g., by injection or transplantation) an effective amount of a composition comprising alginate-coated mesenchymal stem cells comprising a crosslinked alginate hydrogel layer encapsulating single mesenchymal stem cells, wherein the alginate is conjugated at least one cell surface receptor ligand, and wherein the cell surface receptor ligand is unevenly distributed over the surface of each of the alginate-coated mesenchymal stem cells or distributed in predefined locations on the surface of each of the alginate-coated mesenchymal stem cells thereby promoting osteogenesis in the subject.

As used herein, “subject” means an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is preferably a mammal such as a primate or a human.

In some aspects, alginate-coated cells may be administered by engraftment, wherein the cells are injected into the subject, for example, intravenously, intra-muscularly, intra-arterially, intra-bone and the like. In aspects, administration involves providing to a subject about 10², 10⁴, 10⁶, 10⁷, 10⁸, 10⁹, 10¹⁰, 10¹², or more cells. The number of cells engrafted may be chosen based on the route of administration and/or the severity of the condition for which the cells are engrafted.

In additional aspects, alginate-coated cells may be administered in conjunction with a second therapeutic agent, e.g., an agent useful in treating the subject's disease or condition. The second therapeutic agent may be different from the present alginate-coated cells and may include cells, antibodies, proteins and peptides, or small molecules. In some aspects, the second therapeutic agent is selected from ion channel modulators, cell contractility modulators, small GTPase modulators, or a combination thereof. The alginate-coated cells and second therapeutic agent may be administered simultaneously or sequentially. In addition, the alginate-coated cells and second therapeutic agent may be administered from a single composition or two separate compositions. The second therapeutic agent may be administered in an amount to provide its desired effect. The effective dosage range for each second therapeutic agent is known in the art or may be determined by routine experimentation, and the second therapeutic agent may be administered to an individual in need thereof within such established range.

As used herein, the term “amount effective,” “effective amount” or a “therapeutically effective amount” refers to an amount of the cells or composition described herein sufficient to achieve the desired result. The amount of the cells or composition which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. A clinician may titer the dosage or route of administration to obtain the optimal therapeutic effect.

Subjects in need of treatment in accordance with the method herein may include those with a disease or condition who would benefit from the administration of mesenchymal stromal cells. In some aspects, the alginate-coated MSCs are differentiated prior to being administered to the subject. In some aspects, the cells are used to repair tissue in a subject. In accordance with such aspects, the cells form structural tissues, such as bone. In some aspects, the alginate-coated cells are administered to a subject to promote osteogenesis in the subject.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term and “treat” synonyms contemplate administering a therapeutically effective amount of the alginate-coated cells of the invention to an individual in need of such treatment. A treatment can be orientated symptomatically, for example, to suppress symptoms. Treatment can be carried out over a short period, be oriented over a medium term, or can be a long-term treatment, for example within the context of a maintenance therapy.

Also provided herein is a method for preparing the alginate-coated cells described herein. The steps of this method may include (i) providing a first aqueous phase comprising alginate conjugated to a first amount of a first cell surface receptor ligand as described herein; (ii) providing a second aqueous phase comprising unmodified alginate (i.e., alginate that is not conjugated to a cell surface receptor ligand), cells (e.g., a population of hematopoietic cells, immune cells, epithelial cells, endothelial cells, stromal cells, and mesenchymal cells), and a crosslinking agent (e.g., a divalent cation such as Ca²⁺, Sr²⁺ or Ba²⁺); (iii) providing a third aqueous phase comprising unmodified alginate and alginate conjugated to a second amount of the first cell surface receptor ligand or alginate conjugated to a second cell surface receptor ligand; and (iv) simultaneously interfacing the first aqueous phase, second aqueous phase, and third aqueous phase with an oil phase that activates the crosslinking agent, thereby forming droplets comprising the alginate-coated cells described herein, i.e., single cells encapsulated in a layer of crosslinked alginate hydrogel in which the cell surface receptor ligand is unevenly distributed over the surface of each of the alginate-coated cells or distributed in predefined locations on the surface of each of the alginate-coated cells. Advantageously, this method can retain high levels of cell viability without the need to perform additional cell sorting. Moreover, gel thickness and softness may be modulated for different applications. For example, for bone regeneration the gel coating layer thickness may not be greater than 10 microns if softness is 2 kPa, but the thickness may be greater if softness is greater than 10 kPa.

In some aspects, the second aqueous phase comprises unmodified alginate cell-CaCO₃, and cell-SrCO₃, cell-BaCO₃ nanoparticles and the oil phase comprises oil and acetic acid, wherein the acetic acid releases Ca²⁺ or Sr²⁺ or Ba²⁺ from the cell-CaCO₃, cell-SrCO₃, cell-BaCO₃ nanoparticles and wherein the Ca²⁺ or Sr²⁺ or Ba²⁺ crosslinks the alginate in the first aqueous phase, second aqueous phase, and third aqueous phase to form the crosslinked alginate hydrogel layer coating single cells in which the cell surface receptor ligand is unevenly distributed over the surface of each of the alginate-coated cells or distributed in predefined locations on the surface of each of the alginate-coated cells.

In some aspects, the crosslinked alginate hydrogel layer encapsulating single cells is prepared in a microfluidic device, the microfluidic device comprising three microfluidic channels for the first aqueous phase, second aqueous phase, and third aqueous phase, the three microfluidic channels converging on a fourth microfluidic channel for interfacing the first aqueous phase, second aqueous phase, and third aqueous phase with the oil phase; and an outlet channel for conveying the droplets comprising the alginate-coated cells from the fourth microfluidic channel.

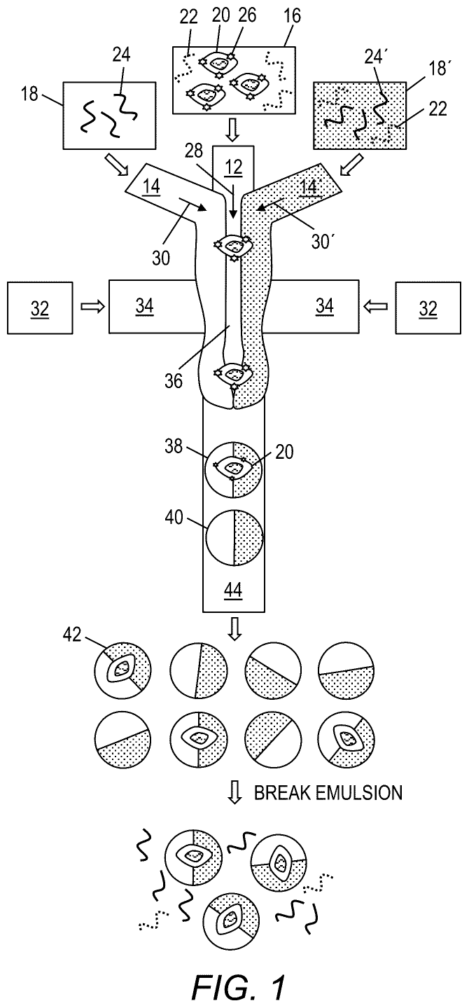

As illustrated in FIG. 1, a device 10 for controlled encapsulation of single cells with a crosslinked alginate hydrogel, wherein the single cells comprise a cell surface receptor ligand unevenly distributed over the surface of each of the cells or distributed in predefined locations on the surface of each of the cells. In some aspects, the device 10 may be composed of three microfluidic channels 12, 14, 14′ for delivering alginate-based aqueous phases 16, 18, 18′, where the composition of each alginate-based aqueous phase 16, 18, 18′ may be independently tuned from each other. The three microfluidic channels 12, 14, 14′ include a middle microfluidic channel 12 and two side microfluidic channels 14, 14′. One side microfluidic channel 14 conveys the flow of a first aqueous phase 18 comprising alginate conjugated to a first amount of a first cell surface receptor ligand 24. The middle microfluidic channel 12 conveys the flow of a second aqueous phase 16 comprising unmodified alginate 22 and cells 20 coated with a crosslinking agent 26. The other side microfluidic channel 14′ conveys the flow of a third aqueous phase 18′ comprising unmodified alginate 22 and alginate conjugated to a second amount of the first cell surface receptor ligand or alginate conjugated to a second cell surface receptor ligand 24′. To focus cells 20 in the middle of the middle aqueous phase 16 in the middle microfluidic channel 12, the flow rate 28 of the middle aqueous phase 20 may be progressively reduced, while the flow rates 30, 30′ of the side aqueous phases 18, 18′ may be increased, while keeping the total aqueous flow rate constant. Microfluidic channels 12, 14, 14′ each interface a junction 36 along with a fourth microfluidic channel 34 which conveys the flow of an oil phase 32 comprising an oil (e.g., a fluorinated oil), a surfactant, and an agent for activating the crosslinking agent 26 (e.g., acetic acid). After emulsification at the junction 36, the droplets 38 that contain cells 20 coated with a crosslinking agent 26 undergo gelation due to activation, while droplets 40 that do not contain the cells 20 coated with a crosslinking agent 26 do not undergo gelation. This results in a pure population of cells in compartmentalized gel coatings 42 that exit the device via outlet channel 44.

The following non-limiting examples are provided to further illustrate the present invention.

### Example 1: Materials and Methods

Cell Culture. D1 mouse MSCs were purchased from American Type Cell Culture (ATCC, CRL-12424). D1 MSCs were cultured in complete high-glucose Dulbecco's Modified Eagle Medium (DMEM, Thermo) supplemented with 1% penicillin-streptomycin (P/S), 1% GlutaMAX™ (Thermo), and 10% fetal bovine serum (FBS, Atlanta Biologicals). Cells were cultured until when they reached ˜80% confluence in a 175 cm² flask by detaching them with trypsin-EDTA (Thermo). D1 MSCs with passage number less than 11 were used in this study.

Alginate Preparation. Sodium alginate (LF200, ˜240 kDa, FMC Biopolymer) was purified by dialyzing against decreasing concentrations of NaCl for 3 days using dialysis membrane (Spectra/Por 3, 3.5 kDa molecular cut-off; Repligen), treated with activated charcoal, and sterile filtered, followed by lyophilization. To introduce an integrin adhesion ligand, an Arg-Gly-Asp peptide (GGGGRGDSP (SEQ ID NO: 5); Peptide 2.0) was covalently conjugated to 1% w/v purified alginate in a 2-morpholinoethanesulfonic acid (MES) buffer (pH 6.5) by 1-ethyl-dimethylaminopropyl (EDC) and N-hydroxysulfosuccinimide (Sulfo-NHS) (Thermo) with a degree of substitution ˜2 (or ˜0.1% of the total number of carboxylic groups per alginate molecule), which was previously shown not to alter E of alginate microgels (Wong et al. (2020) Adv. Sci. (Weinh) 7:2001066). After dialysis, the concentration of RGD conjugated to alginate (˜60 μM at 1% w/v) was verified by the Lavapep® Fluorescent Protein and Peptide Quantification Kit (Gel Company) according to the manufacturer's instructions.

Microfluidic Device Fabrication. Microfluidic devices were produced by soft lithography (Qin, & Xia (2010) Nature Protocols 5:491). A negative photoresist SU-8 25 (Viscosity: 2500 cSt, Kayaku Advanced Materials) was spin-coated onto a clean silicon wafer (University Wafer) with a defined height, followed by UV exposure through a transparency photomask (CAD/Art Services) for patterning (FIG. 1). After developing the mold, polydimethylsiloxane (PDMS) (Dow Corning) was mixed with a crosslinker at ratio 10:1, degassed, poured, and cured for at least 3 hours at 65° C. The cured PDMS was then peeled off the mold and bonded to an oxygen-plasma treated glass slide for 1 hour at 65° C. Microfluidic channels were treated with AQUAPEL® glass treatment (PPG Industries) and dried to make them hydrophobic for emulsification. To connect syringes (BD) to microfluidic channels, polyethylene tubing (inner diameter: 0.38 mm; outer diameter 1.09 mm) and 27G×½ needed were used. Syringe pumps (Harvard Apparatus) were used to individually control aqueous and oil flow rates.

Single Cell Encapsulation in Compartmentalized Microgels. CaCO₃ nanoparticles (CalEssence; 900 nm diameter) were resuspended in complete DMEM medium and dispersed by sonication with Vibra Cell Sonicator for 1 minute at 75% amplitude. The nanoparticles were then centrifuged for 5 minutes at 50 g to remove aggregated particles, followed by centrifugation for 5 minutes at 1000 g for collection. Monodispersed CaCO₃ nanoparticles were resuspended with serum-free DMEM medium. The concentration of purified CaCO₃ nanoparticles was decreased from 24 mg/mL to 18 mg/mL with smaller alginate gel coatings with E˜2 kPa, and further down to 5 mg/ml for softer (E˜0.5 kPa) gel coatings. Cells were then incubated with CaCO₃ by rotation for 1 hour at room temperature. Excess CaCO₃ nanoparticles were washed out by centrifugation for 5 minutes at 50 g. Three aqueous phases (one middle and two side channels) were prepared with 1% w/v alginate solution in the buffer composed of DMEM with 50 mM HEPES, 10% FBS, 1% P/S at pH 7.8. In the middle aqueous phase, CaCO₃-coated cells were added. In some experiments, cells were labeled with 2 μM calcein AM (Biotium) to test cell viability during the encapsulation process. In each of the aqueous phases in the two side channels, a small amount (final w/v=0.05%) of 10/60 alginate (˜120 kDa; FMC Biopolymer) conjugated with either Lissamine™ rhodamine B ethylenediamine (Thermo) or CF™ 647-amine (Sigma) was added to visualize each gel compartment. Alginate-RGD was added to either one compartment or both compartments for asymmetric and symmetric RGD presentation, respectively. The oil phase was based on fluorinated oil (HFE-7500; 3M) mixed with 3% (for large droplets) or 1% small (for droplets) V/V perfluoropolyether (PFPE, Krytox; Miller Stephenson) as a surfactant, and 0.03% v/v acetic acid as an initiator of Ca²⁺ release from CaCO₃ nanoparticles. The channel dimensions of the microfluidic device (height×width in μm) were: 70×70 and 20×30 for large and small droplets, respectively. To focus cells in between the two side compartments, the aqueous flow rates were varied in three steps where the side flow rates were progressively increased and the middle flow rate was decreased, while keeping the total aqueous flow rate constant. For large droplets, the total aqueous flow rate was kept at 1.65 μL/min, while the side flow rates were varied from 0.2 μL/min to 1.55 L/min (the middle flow rate from 1.45 μL/min to 0.1 μL/min). For small droplets, the total aqueous flow rate was kept at 1 μL/min, while the side flow rates were varied from 0.12 μL/min to 0.94 μL/min (the middle flow rate from 0.88 L/min to 0.06 μL/min). The flow rate of the oil phase was kept 5 times higher than the total aqueous phase. Each step was run for 10 minutes before moving to the next step. An inverted microscope (Nikon) with a high-speed camera (Zyla 4.2 sCMOS, Andor) was used to monitor and analyze laminar flow, droplet formation, and cell distribution in the microfluidic device. The emulsion for downstream analyses was collected every 20 minutes, followed by 20 minutes rotation at room temperature. The emulsion was then broken by adding 10% 1H, 1H, 2H, 2H-perfluorooctanol (Alfa Aesar). Hydrogel-coated cells were washed twice with serum-free DMEM. Unless otherwise specified, ˜10,000 hydrogel-coated cells were embedded in 50 μL of 1.25 mg/mL collagen-I matrix (Rat tail; Thermo) on a 48-well glass bottom plate (MatTek), followed by culture at 37° C. in complete DMEM for downstream analyses.

Cell Encapsulation in Bulk Alginate Hydrogels. Cells were resuspended in 1% w/v alginate-RGD (˜60 μM) in serum-free DMEM, and rapidly mixed with CaSO₄ by syringes with a Luer-Lok connector. The final concentration of 10 mM CaSO₄ was used to form the bulk hydrogel with E˜2 kPa (Wong et al. (2020) Adv. Sci (Weinh) 7:2001066; Wong et al. (2022) Nat. Biomed. Engineer. 6:54). The mixed solution was deposited between two glass plates with a 1 mm void thickness. After 1.5 hours, hydrogels were punched into discs with a 5 mm diameter and cultured on a 96-well glass bottom plate (MatTek) in complete DMEM.

Mechanical Characterization of Compartmentalized Hydrogel Coatings around Single Cells. A glass slide was coated with 0.1 mg/mL of poly-L-lysine (Sigma) for 2 hours. After washing out poly-L-lysine, hydrogel-coated cells were immobilized on the glass slide for 1 hour and rinsed with DMEM. The slide was then transferred to an MFP-3D-BIO system (Asylum Research) to perform atomic force microscopy with a silicon nitride cantilever with an 18° pyramid tip (MLCT, Bruker). A cantilever spring constant was determined from thermal fluctuations at room temperature (˜40 mN/m) before each analysis. A fluorescent microscope was used to bring the cantilever to the surface of each fluorescently labeled hydrogel compartment. Indentation was then performed under contact mode with 1 μm/s velocity and force distance 500 nm until the trigger deflection voltage (0.5 V) was reached. To calculate Young's modulus (E), force-indentation curves were fitted to the Hertzian model with a pyramid indenter and Poisson's ratio (ν)=0.5.

Confocal Microscopy for 3D Image Analysis. Hydrogel-coated cells were incubated with 2 μM of calcein AM and 1 μM of Hoechst 33342 (Thermo) for 1 hour to stain cytoplasm and nucleus, respectively. Samples were then washed with HBSS and maintained in Fluorobrite™ DMEM (Thermo) at 37° C. and 5% CO2 in the Zeiss LSM 770 confocal microscopy system with a motorized stage and the 20×/0.8 M27 Plan-Apochromat objective. To analyze cell volume and sphericity, z-stacks were captured with 50-80 μm total depth with each image at 0.77 μm for 65-100 images per z-stack. The stacks were analyzed in Imaris software (Bitplane, version 7.7.2) to perform 3D reconstruction. Voxels were generated for each gel compartment in red (rhodamine) or blue (CF™ 647), green (calcein), and cyan (Hoechst) signals after automatic thresholding with 10% variation across all the images from different experiments. A hydrogel-coated cell was considered an outlier if it met one of the following exclusion criteria: (1) It touches another hydrogel-coated cell, (2) Cyan voxels extend beyond the green voxel boundary, (3) Green and cyan voxels are not within hydrogel voxels, or (4) Hydrogel voxels do not contain green or cyan voxels inside. The total voxels above the threshold were then calculated to quantify hydrogel, cytoplasmic, and nuclear volumes of each hydrogel-coated cell. Sphericity of cell and nucleus was analyzed from the same set of voxels and defined as (π_{1/3} (6V)^{2/3})/A, where V is volume and A is surface area. The contact area between the cell and each hydrogel compartment was estimated by the built-in algorithm (XTension) in Imaris software.

Inducible Lentiviral Expression of F-tractin Fused with tdTomato. To visualize actin polymers in live cells after encapsulation in hydrogel coatings while minimally impacting baseline cellular functions, the plasmid containing F-tractin fused with the fluorescent gene tdTomato (a gift from Andrei Karginov, UIC) was cloned into the lentiviral vector pcW57.1 (Addgene plasmid #41393) for inducible expression in the presence of doxycycline. F-tractin is known to selectively bind to polymerized F-actin (Johnson & Schell (2009) Molec. Biol. Cell 20:5166). Lentiviral particles were produced with a second-generation lentiviral packaging system (LV003, Applied Biological Materials) using a transfection reagent (Lentifectin™, Applied Biological Materials) in HEK293T cells. Lentiviral particles were purified and applied to D1 MSCs at passage 5 with polybrene (8 μg/mL; Sigma) for 3 days, followed by selection of transduced cells by puromycin (5 μg/mL; Sigma) for 7 days and sorting of tdTomato⁺ cells after doxycycline (500 ng/ml; Cayman Chemical) treatment for 1 day.

Confocal Analysis of Cell Membrane and F-actin in Live Cells. To visualize the cell membrane, MSCs were labeled with 5-hexadecanoylaminofluorescein (HEDAF, 0.5 mg/mL; Thermo) prior to encapsulation in microgels composed of red (rhodamine) and blue (CF™ 350) compartments. To visualize F-actin, F-tractin-tdTomato D1 MSCs were treated with doxycycline (500 ng/ml) for 1 day to induce the expression of F-tractin-tdTomato prior to encapsulation in microgels composed of far red (CF™ 647) and blue (CF™ 350) compartments. After 1 day in culture, confocal analysis was done with the 63×/Plan-Apochromat 1.46NA oil objective at 37° C. and 5% CO2. A z-stack of images was obtained for each cell in a microgel and rotated in Imaris software so that the plane that divided the two gel compartments could be oriented vertically. The horizontal plane perpendicular to the vertical plane was drawn in the middle height of the cell to obtain the midplane. The projected image on the midplane was mapped to each gel compartment and used for subsequent analysis by using ImageJ (2.1.0, National Institutes of Health).

Measurement of Cell Membrane-RGD Interactions by Förster Resonance Energy Transfer (FRET) Detected by Fluorescence Lifetime Imaging Microscopy (FLIM). Cells (4 million/mL) were labeled with the membrane dye HEDAF (0.5 mg/mL) for 1 hour in 37° C. as a donor, followed by encapsulation in microgels composed of red (tetramethylrhodamine (TAMRA)-conjugated RGD with a degree of substitution ˜2, acceptor) and blue (CF™ 350) compartments. FLIM was then performed to quantify the reduction in donor fluorescence due to acceptor quenching upon FRET, by using the Ultima Multiphoton Microscope System equipped with a Becker and Hickl time-correlated single-photon counting module (Bruker). HEDAF was excited at 820 nm by the Chameleon Ultra II Two-Photon laser operating at 80 MHz. The emission signal was collected through a 595/60 nm shortpass filter for 30 seconds. Signal decay time (τ) values were extracted by fitting the average photon count vs. time graph to a two-phase exponential decay fit in the data analysis software SPCImage (Becker & Hickl GmbH). The first component of the lifetime in the curve fit was reported in this study, since it accounts for the majority of the signal.

Measurement of Membrane Tension by FLIM. Cells in hydrogels were incubated with 1 μM of Flipper-TR® lipid membrane tension probe (Cytoskeleton, Inc.) (Colom et al. (2018) Nat. Chem. 10:1118) in complete DMEM for 30 minutes. One of the hydrogels was labeled with red (rhodamine) fluorescence to distinguish between two compartments. FLIM was performed by exciting the probe at 920 nm, 80 MHz. The emission signal was collected through a 595/50 nm bandpass filter for 1 minute, followed by extraction of τ values using SPCImage.

Quantification of Rho GTPase Activity by FRET Detected by FLIM. To quantify Cdc42 or Rac1 activity in live MSCs in hydrogels, MSCs were transfected with plenti-Cdc42-2G (Addgene plasmid #68813) or pLenti-Rac1-2G (Addgene plasmid #66111), respectively. Each plasmid (4 μg) and 1 million cells were mixed with 100 μL of NUCLEOFECTOR® transfection solution from the NUCLEOFECTOR® kit (Lonza) and electroporated using a high-viability program (C-17) in Amaxa (Lonza). Transfected cells were cultured on plastic overnight. After encapsulating transfected cells in microgels and cultured for an additional day, FLIM was performed by exciting the donor (mTFP1-WASP CRIB for Cdc42, mTFP1-PBD for Rac1) at 860 nm, 80 MHz. The emission signal was collected through a 595/60 nm shortpass filter for 1 minute, followed by extraction of t values using SPCImage.

Cell Retrieval from Hydrogels. Cells in hydrogels were retrieved by digesting with alginate lyase (4 mg/mL; Sigma), collagenase P (2.5 mg/mL; Sigma), and trypsin-EDTA (0.125%; Thermo) for 30 minutes at 37° C. Samples were then centrifuged at 3000 rpm for 5 minutes, and washed twice with HBSS.

MSC Differentiation and Assays. MSCs in hydrogels were cultured for 1 day in a basal medium (No. CCM007), followed by the medium supplemented with both osteogenic (No. CCM007) and adipogenic (No. CCM11) cocktails for 10 days. The medium was refreshed on day 5. All reagents for MSC differentiation were purchased from R&D Systems. One half of each sample was used to quantify an absolute number of viable cells by calcein staining, while the other half was used to evaluate early osteogenesis or adipogenesis by ALP activity or lipid droplet staining, respectively. To quantify ALP activity, cells were lysed with 200 μL passive buffer (No. E1941, Promega) for 15 minutes at 4° C. Each lysate was then added to a black 96-well plate preloaded with 100 μL 4-methylbelliferyl phosphate (4-MUP) substrate (Sigma). Signals were acquired with excitation at 360 nm and emission at 450 nm using a plate reader. Recombinant mouse ALP protein (Novus Biologicals) was used to generate a standard curve. To quantify lipid droplets in cells, cells were incubated with the LipidSpot™ 610 Lipid Droplet Stain (Biotium) at 37° C. for 30 minutes. Signals were acquired with excitation at 592 nm and emission at 638 nm using a plate reader.

Gene Expression Analysis. Cells were lysed with Trizol™ reagent (500 μL; Thermo) for 10 minutes. Samples in Trizol™ reagent were stored at −80° C. for up to one week before processing. Chloroform (100 μL) was added for phase separation. Samples were centrifuged for 10 minutes at 12,500 rpm, 4° C. The top layer containing RNA was collected into a new tube, and then precipitated with isopropanol (1 mL) for at least 15 minutes at 4° C. Samples were then centrifuged at 12, 500 rpm for 10 minutes at 4° C. The supernatant was removed, and the precipitated RNA was washed with 75% ethanol, followed by centrifugation for 5 minutes at 7500 rpm, 4° C. After removing ethanol, purified RNA was resuspended in 15 μL of RNase-free water (Thermo). NanoDrop spectrophotometer (Thermo) was used to quantify RNA concentration and quality. CDNA was obtained by reverse transcription using SUPERSCRIPT®-III reverse transcriptase (Thermo). For each sample, 50 ng cDNA was added to each well in triplicate, followed by the Power SYBR™ Green PCR Master Mix (Applied Biosystems). Quantitative PCR was performed in the ViiA™ 7 qPCR system (Thermo). Relative gene expression was calculated using the 2^{-ΔCt} method by normalizing the cycle threshold (Ct) value of each target gene to that of the reference gene (Gapdh). Primer sequences are provided in Table 1.

TABLE 1

| Gene | | | |
|---|---|---|---|
| (GENBANK | | | SEQ |
| Accession | | | ID |
| No.) | Primer | Sequence (5′→3′) | NO: |
| Gapdh | Forward | CTTTGTCAAGCTCATTTCCTGG | 30 |
| (NM_008084. | Reverse | TCTTGCTCAGTGTCCTTGC | 31 |
| 3) | | | |
| Alp | Forward | CTCCAAAAGCTCAACACCAATG | 32 |
| (NM_007431. | Reverse | ATTTGTCCATCTCCAGCCG | 33 |
| 3) | | | |
| Runx2 | Forward | GCTATTAAAGTGACAGTGGACGG | 34 |
| (NM_009820) | Reverse | GGCGATCAGAGAACAAACTAGG | 35 |
| Pparg1 | Forward | TGTTATGGGTGAAACTCTGGG | 36 |
| (NM_011146) | Reverse | AGAGCTGATTCCGAAGTTGG | 37 |
| Cdc42 | Forward | CATGTCTCCTGATATCCTACACAAC | 38 |
| (NM_009861) | Reverse | TGTCATAATCCTCTTGCCCTG | 39 |
| Rac1 | Forward | TGCTTTTCCCTTGTGAGTCC | 40 |
| (NM_009007) | Reverse | TCAGCTTCTCAATGGTGTCC | 41 |

Chemical Inhibitors. The following chemical inhibitors were purchased from Cayman Chemical: Cilengitide, NSC23766, and ML141. Rhosin was purchased from R&D Systems. DMSO was used as a negative control and purchased from Sigma.

RNA Interference. Small interfering RNAs (siRNAs) for Cdc42 and Rac1 were purchased from Horizon Discovery. Scrambled siRNA control (Silencer negative control no. 1) was purchased from Thermo. SiRNA (4 nM) was mixed with LIPOFECTAMINER RNAiMAX transfection reagent (Thermo) for 20 minutes in Opti-MEM™ (Thermo). The mixture was then applied to cells and cultured for 3 days before cell encapsulation in hydrogels. qPCR was used to confirm the knockdown efficiency of each target gene compared to the scrambled control.

Finite Element Analysis to Model Gel Stress. A finite element model was implemented using the commercial finite element package Abaqus Standard to solve the linear elastic Eshelby's inclusion problem (Eshelby (1957) Proc. Royal Soc. A 1241:376): an expanding cell inclusion n inside the hydrogel. The model was formulated as an axisymmetric 3D geometry for its symmetry with respect to the horizontal axis. Both the cell and the hydrogel were defined as simple linear elastic, incompressible (Poisson's ratio v=0.5) materials. The Young's modulus (E) of both the hydrogel and the cell was set to 1500 Pa based on the experimental data and previously determined values (Wong et al. (2020) Adv. Sci. (Weinh) 7:2001066). The model was simulated with a mesh of 13,000 elements, in which the convergence was achieved.

To solve this inclusion problem, a non-uniform isotropic transformation volume expansion (eigenstrain) (Eshelby (1957) Proc. Royal Soc. A 1241:376) was imposed to simulate cell volume expansion in response to asymmetric ligand (RGD) presentation. Then, the final deformation and stress values of both the cell and the hydrogel were calculated by considering the constraint of the cell expansion due to the hydrogel in finite element analysis. The transformation cell volume expansion was defined based on a quadratic equation which minimizes the expansion of the cell at its center to zero and assigns asymmetric transformation values to the far edges on the horizontal axis. The quadratic distribution of the transformation axial strain as a result of cell volume elongation is expressed as follows

$${ϵ}_{T} = ( \frac{{ϵ}_{{RGD}^{1}} + {ϵ}_{{RGD}^{0}}}{2 {R}^{2}} ) {X}^{2} + ( \frac{{ϵ}_{{RGD}^{1}} - {ϵ}_{{RGD}^{0}}}{2 R} ) X$$

where R is the initial radius of an MSC (˜7.81 μm^{[24]}), X is the coordinate along the horizontal axis, with the origin at the center of the undeformed cell with the range between −R and R, and ϵ_{RGD}_{¹} and ϵ_{RGD}_{⁰} are the maximum transformation strains in the axial direction towards the RGD and RGD-null sides, respectively.

With ϵ_{RGD}_{¹}=0.882 and ϵ_{RGD}_{⁰}=0.573, the maximum horizontal displacement of the cell toward the RGD side is 2.7 μm, the maximum horizontal displacement toward the RGD-null side is 1.6 μm, and the maximum vertical displacement is 0.65 μm for each of upper and lower directions. These values closely mirror the experimental results (FIG. X 4C).

Statistical Analysis. Statistics were performed as indicated using GraphPad Prism version 9.3.1. Unless otherwise noted, statistical comparisons were made by one-way analysis of variance (ANOVA) followed by Tukey's multiple comparisons test when standard deviations did not vary between experimental groups, and Welch's ANOVA, followed by Dunnett T3 multiple comparisons test when standard deviations were variable. For nested datasets, such as cell volume and sphericity of individual cells in response to drugs from each experiment, nested one-way ANOVA followed by Tukey's test was performed. A p-value less than 0.05 established statistical significance.

### Example 2: Deterministic Single Cell Encapsulation in Microgels with Tunable Spatial Presentation of Ligands

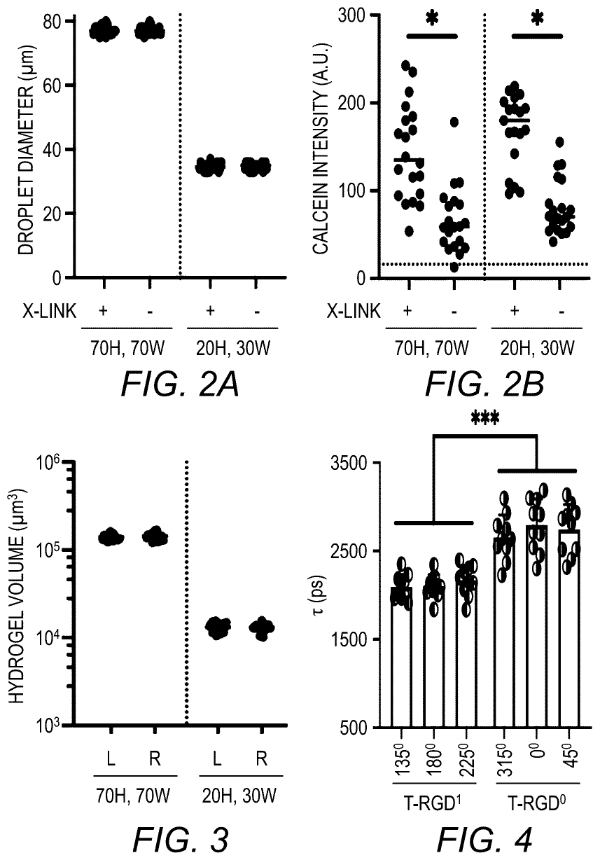

To control the spatial presentation of ligands around single cells in a 3D space, a droplet-based microfluidic approach was developed to prepared a pure population of single cells that are placed between two distinct compartments (‘Janus’) in microgels. To achieve this, a microfluidic device was designed with three distinct input channels (two side channels and one middle channel) for aqueous phases that merge at the junction prior to droplet formation (FIG. 1). Devices with two different dimensions (height, width in μm=70 h, 70 w or 20 h, 30 w) were fabricated to test whether distinct compartments can be formed within single cell-encapsulating microgels with varied sizes. High viscosity alginate (LF200, ˜240 kDa, viscosity=200-400 mPa·s, 1% w/v) was chosen as a base material for all of the aqueous phases to minimize the possibility of mixing. A small fraction of fluorescently-conjugated alginate was added as a model hydrogel-bound ligand to visualize each aqueous phase in the side channels. The aqueous phase maintains laminar flow not only at the junction but also when the oil phase is about to pinch off the aqueous phase into droplets. To place single cells between two compartments, cells were added to the middle channel. MSCs were chosen as a model cell type to study cell-matrix interactions. Clonally-derived murine D1 MSCs (˜15.6 μm in diameter or ˜2000 μm3 in total cell volume) were used, since they are known to show less cell-to-cell heterogeneity compared to primary cells. The flow rate of the middle aqueous channel was then progressively reduced, and that of the side channels was increased, while the total aqueous flow rate was kept constant. This process not only focused cells in the middle of the aqueous phase at the junction but also allowed cells to contact with the side aqueous phases. Cells remained localized in the midline of the droplets even after droplet formation without alginate crosslinking. However, in order to purify single cells in microgels, cells were coated with CaCO₃ nanoparticles and the excess was washed out so that the gelation of alginate could occur only when droplets contained cells (Mao et al. (2017) Nat. Mater. 16:236; Wong et al. (2020) Adv. Sci. (Weinh) 7:2001066). The cell concentration was kept at ˜15 million per mL in the middle aqueous phase to maximize the number of purified cells in microgels, while keeping the average of one cell per microgel (˜90% of the total population). While the size of single cell-containing droplets depended on the device dimensions (FIG. 2A), immediate crosslinking of alginate upon droplet formation by adding acetic acid in the oil phase to release Ca²⁺ from CaCO₃ on the cell surface was required to maintain cell viability as indicated by calcein staining (FIG. 2B). To this end, this method resulted in a purified population of single cells localized in the middle of Janus microgels with varied sizes (FIG. 3), and each cell contacted with both compartments to an equal extent. In addition, Young's modulus (E) of each hydrogel compartment remained the same at ˜2 kPa, and was tunable down to ˜0.5 kPa by lowering the CaCO₃ concentration, the values that are exhibited by natural soft tissues. Thus, this method may be used to present hydrogel matrix-bound ligands to single cells either symmetrically or asymmetrically independently of base polymer, gel size, and gel elasticity.

### Example 3: Controlling the Spatial Presentation of Integrin Ligands Around Single Cells in a 3D Space

The method for preparing hydrogel encapsulated single cells was leveraged to understand cellular responses to asymmetric cell-matrix adhesion at the single cell level in a 3D space. The RGD ligand was chosen as an integrin ligand due to its ability to interact with a number of integrins, including α₅β₁ and αᵥβ₃. To quantify the interaction between cell membrane and RGD in the gel, a Forster Resonance Energy Transfer (FRET)-based approach was used by labeling the cell membrane of MSCs with a fluorescein-based dye (donor) and conjugating the tetramethylrhodamine (TAMRA)-labeled RGD (T-RGD) peptide (acceptor) to alginate (Kong et al. (2006) Proc. Natl. Acad. Sci. USA 103:18534). Here, fluorescence lifetime imaging microscopy (FLIM) was used, since it enables FRET measurements independently of fluorophore concentrations. With FRET, the donor emission becomes quenched, resulting in a decreased fluorescence lifetime (τ). Encapsulating fluorescein-labeled cells in microgels with T-RGD decreased τ, while treatment with cilengitide, a soluble cyclic RGD peptide that competitively inhibits integrins, restored τ. Thus, decreased t was selectively due to integrin-RGD interactions. Importantly, subcellular analysis showed that τ was decreased only on the side of single cells that were in contact with T-RGD when added to one of the compartments in Janus microgels (FIG. 4). Thus, this approach may be used to control the polarity of integrin-ligand interactions in a 3D hydrogel matrix around single cells.

### Example 4: Accelerated Elongation of Single Cells by Asymmetric Presentation of Integrin Ligands

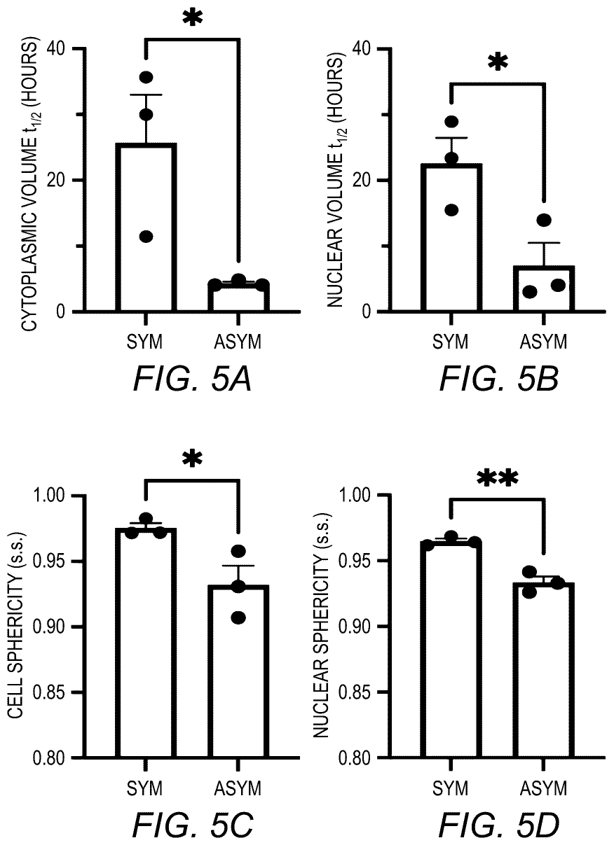

To understand how the polarity of cell-matrix interactions impacts single cell morphology and volume in a 3D space, single MSCs were encapsulated in microgels with either symmetric or asymmetric RGD presentation. A thinner gel (˜2.3×10⁴ μm3 in total volume or ˜10 μm in thickness, FIG. 3) was chosen to encapsulate single MSCs for downstream analyses. MSCs in the microgels were subsequently embedded in a collagen-I gel at a low density (˜10,000 cells in 50 μL), labeled with calcein or Hoechst to visualize the cytoplasm and the nucleus, respectively, followed by confocal imaging analysis to evaluate their volume changes over time in a 3D space. Without RGD in microgels, the volume of the cytoplasm and nucleus was previously determined to be approximately 1000 μm³ each for mouse MSCs (Wong et al. (2020) Adv. Sci. (Weinh) (7:2001066). With symmetric RGD presentation, MSCs expanded in volume over 3 days by ˜1.5-fold but remained spherical, suggesting isotropic expansion. Increasing the total RGD concentration by 2-fold in both compartments did not impact cell volume expansion kinetics or sphericity. In contrast, when RGD was asymmetrically distributed, MSCs expanded in volume faster, while both cell sphericity and nucleus sphericity decreased over time (FIGS. 5A-5D), indicating cell elongation. Hydrogel volume of both compartments remained unchanged over 3 days in culture. Using an intermediate ligand ratio between the two compartments (Left:Right=0.75:0.25) showed the progressive acceleration in cell volume expansion and decrease in sphericity from symmetric to asymmetric RGD presentation. Consistent with previous studies (Mao et al. (2017) Nat. Mater. 16:236; Wong et al. (2020) Adv. Sci. (Weinh) 7:2001066; Wong et al. (2022) Nat. Biomed. Engineer. 6:54), most mouse MSCs in the high molecular weight alginate hydrogel with asymmetric RGD presentation did not proliferate over 3 days in culture, as confirmed by a low level of 5-ethynyl-2′deoxyuridine (EdU) incorporation into the nucleus for 3 days, as opposed to most MSCs in collagen-I gel, which incorporated EdU within 1 day in culture. Lowering gel elasticity to ˜0.5 kPa did not increase the proliferation of encapsulated MSCs. Together, polarized cell-matrix interactions increased the probability of single cells to undergo volume expansion by elongation.

### Example 5: Polarized Membrane Tension in Single Cells by Asymmetric Presentation of Integrin Ligands

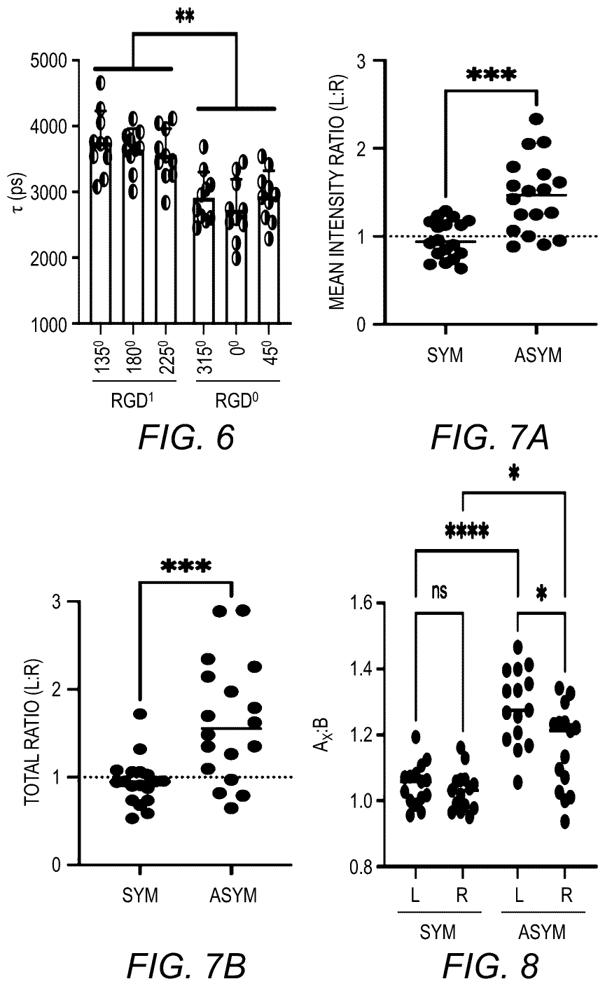

Cell volume expansion is known to result in increased membrane tension as a function of osmotic pressure and cortical contractility. To understand how cell elongation influences membrane tension in response to polarized cell-matrix interactions, a chemical tension reporter was used that increases τ under FLIM in response to higher membrane tension (Colom et al. (2018) Nat. Chem. 10:1118). MSCs in the microgels showed progressive increases in average membrane tension per cell from symmetric to asymmetric RGD presentation after 1 day in culture, which correlated with faster cell volume expansion kinetics. Membrane tension remained higher on the side of single MSCs in contact with RGD (FIG. 6), indicating that membrane tension became polarized with asymmetric RGD presentation. In contrast, membrane tension was uniform with more symmetric RGD presentation or in bulk gels. Adding a small fraction ( 1/20) of alginate-rhodamine to delineate either RGD¹ or RGD⁰ compartment did not change t values. Polarized membrane tension was associated with changes in cytoskeletal structures where the actin cortex in live cells was assembled preferentially toward the RGD-presenting compartment (FIGS. 7A-7B). The confocal analysis of the cell membrane relative to each compartment on the midplane showed that MSCs with asymmetric RGD presentation elongated in both directions but the elongation was significantly higher toward the RGD-presenting compartment (FIG. 8). A model based on finite element analysis showed that non-uniform isotropic volume expansion was sufficient to recapitulate the experimental results where an MSC (with the radius prior to expansion ˜7.81 μm) elongated greater toward the RGD side than the RGD-null side from the center, resulting in a gradient of tension. Thus, asymmetric cell-matrix adhesion polarized membrane tension in single cells during the cell elongation process.

### Example 6: Lineage Specification of Single MSCs by Varying the Spatial Presentation of Integrin Ligands

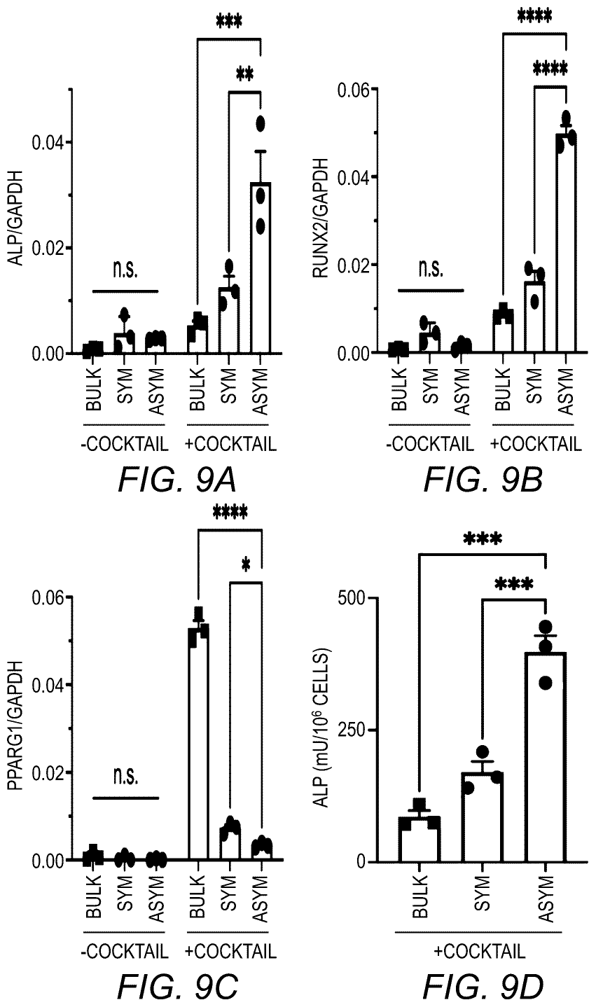

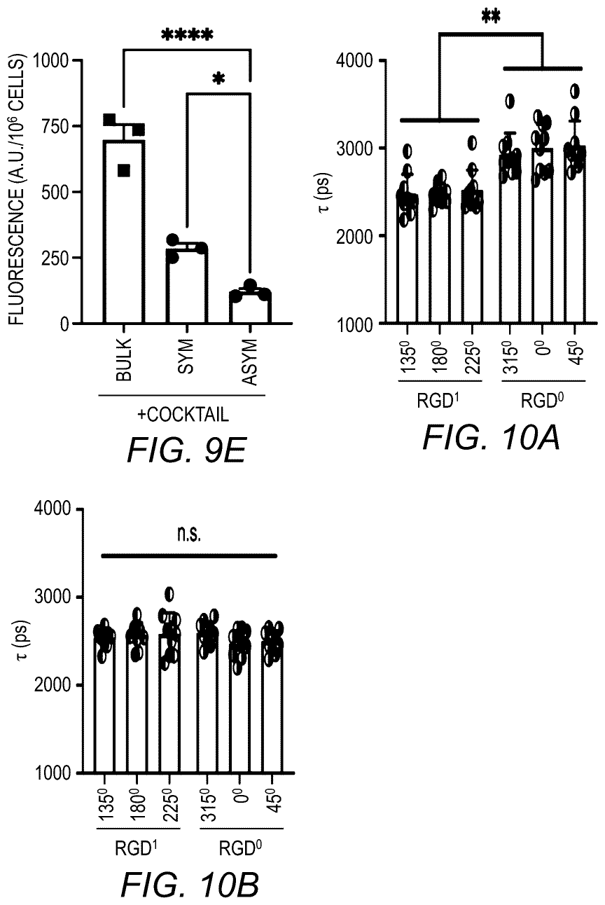

Increased cell spreading and generation of traction forces in engineered 3D matrices have generally been linked to the commitment of MSCs toward osteogenic lineages (Khetan et al. (2013) Nature Materials 12:458; Lee et al. (2019) Nat. Commun. 10:529). It was tested whether cell elongation with polarized membrane tension was associated with lineage specification of MSCs in a longer time scale. MSCs in the microgels with symmetric or asymmetric RGD presentation were cultured for 10 days in the absence or the presence of both osteogenesis- and adipogenesis-promoting cocktails. Most MSCs in the microgels with asymmetric RGD presentation did not proliferate and remained viable in this culture condition. These groups were compared with the bulk alginate-RGD hydrogel (E˜2 kPa). Without the chemical cocktails, no differentiation was observed across all the tested groups. In the presence of the chemical cocktails, MSCs in the microgels with asymmetric RGD presentation showed significantly higher gene expression levels of osteogenic markers, including Alp and Runx2 (FIG. 9A). In contrast, MSCs in the microgels with symmetric RGD presentation or the bulk gel showed a higher level of an adipogenic marker, Pparg1 (FIG. 9B). Alkaline phosphatase (ALP) enzyme activity assay (FIG. 9C) and fluorescent lipid droplet staining (FIG. 9D) confirmed the osteogenic commitment of multipotent MSCs with asymmetric cell-matrix adhesion. MSCs were committed less toward osteogenic lineages but more toward adipogenic lineages in bulk gels than symmetric microgels due to a higher amount of hydrogel per cell. Thus, the symmetry of cell-matrix interactions mediated the lineage specification of single stem cells.

### Example 7: Cdc42 is Essential for Single MSCs to Respond to Asymmetric Presentation of Integrin Ligands

To understand what mediates the cellular responses upon asymmetric cell-matrix interactions, the role of Rho family GTPases was tested, since these enzymes are known to be involved in cytoskeleton remodeling and cell polarity. To evaluate the activity of Cdc42 and Rac1 in live cells, FRET-based biosensors (Martin et al. (2015) Dev. Cell 35:78) were introduced to MSCs by nucleofection prior to encapsulation in the hydrogel. FLIM was used to measure t values of interacting monomeric teal fluorescent protein (mTFP1)-tagged membrane proteins (Wiskott-Aldrich syndrome protein (WASP) Cdc42/Rac interacting binding (CRIB) domain for Cdc42, p21-activated kinase 1 binding domain (PBD) for Rac1; donor), which are decreased when Venus fluorescent protein-tagged Cdc42 or Rac1 (acceptor) reach the membrane during the activation process (Hodge & Ridley (2016) Nat. Rev. Molec. Cell Biol. 17:496). Subcellular analysis showed that t values of the Cdc42 biosensor were significantly lower on the side of single cells that were in contact with RGD (FIG. 10A). In contrast, t values of the Rac1 biosensor remained constant across the cell membrane regardless of the presence of RGD (FIG. 10B). Thus, Cdc42 activity in single cells became polarized in response to asymmetric cell-matrix adhesion.

To test whether Cdc42 mediates the cellular responses to asymmetric cell-matrix interactions, the effects of inhibitors against Rho GTPases were tested. After encapsulation of single MSCs in the microgels with asymmetric RGD presentation, they were cultured for 1 day to reach the steady state in cell volume expansion and elongation, followed by 2-hour treatment with inhibitors against Rho GTPases for downstream analyses. ML141 (Cdc42 inhibitor) but not NSC23766 (Rac1 inhibitor) and Rhosin (RhoA inhibitor) reduced both cytoplasmic and nuclear volumes while turning cells and nuclei back to the spherical shape, indicating that Cdc42 is required for cell elongation. Importantly, ML141, but not other drugs, reduced overall membrane tension, while equalizing membrane tension across both microgel compartments. To validate these results with small molecule inhibitors, MSCs were treated with small interfering RNA (siRNA) against Cdc42 or Rac1 prior to encapsulation, which led to approximately a 70% decrease in target gene expression. Consistently, the knockdown of Cdc42 but not Rac1 reversed the observed changes in cell volume and membrane tension in response to asymmetric RGD presentation. In addition, ML141 inhibited osteogenic differentiation while promoting adipogenic differentiation. Together, the results highlight Cdc42 as a unique mediator of cell volume expansion by elongation, the process that is linked to polarization of membrane tension and lineage specification in response to polarized cell-matrix interactions.

What is claimed is:

1. Alginate-coated cells comprising a crosslinked alginate hydrogel layer encapsulating single cells, wherein the alginate is conjugated to at least one cell surface receptor ligand, and wherein the at least one cell surface receptor ligand is unevenly distributed over the surface of each of the alginate-coated cells or distributed in a predefined location on the surface of each of the alginate-coated cells.

2. The alginate-coated cells of claim 1, wherein the alginate-coated cells are elongated relative to alginate-coated cells in which the cell surface receptor ligand is evenly distributed over the surface of the alginate-coated cells.

3. The alginate-coated cells of claim 1, the crosslinked alginate hydrogel layer encapsulating the single cells has a thickness of about 0.5 microns to about 30 microns and a softness of about 0.1 kPa to about 10 kPa, and the alginate has a molecular weight of about 250 kDa to about 500 kDa.

4. The alginate-coated cells of claim 1, wherein the at least one cell surface receptor ligand is a polysaccharide and/or peptide/protein.

5. The alginate-coated cells of claim 1, wherein the crosslinked alginate hydrogel layer is crosslinked with Ca²⁺, Sr²⁺, or Ba²⁺.

6. The alginate-coated cells of claim 1, wherein the alginate-coated cells are capable of undergoing cell polarization.

7. The alginate-coated cells of claim 6, wherein the alginate-coated cells are selected from the group consisting of hematopoietic cells, immune cells, epithelial cells, endothelial cells, stromal cells, and mesenchymal cells.

8. The alginate-coated cells of claim 7, wherein the mesenchymal cells are mesenchymal stem cells.

9. The alginate-coated cells of claim 1, wherein the crosslinked alginate hydrogel layer further comprises one or more growth factors.

10. A composition comprising the alginate-coated cells of claim 1 and a pharmaceutically acceptable carrier or aqueous medium.

11. The composition of claim 10, further comprising one or more ion channel modulators, cell contractility modulators, small GTPase modulators, or a combination thereof.

12. A method of promoting osteogenesis in a subject in need of treatment comprising administering to the subject an effective amount of a composition comprising alginate-coated mesenchymal stem cells comprising a crosslinked alginate hydrogel layer encapsulating single mesenchymal stem cells, wherein the alginate is conjugated to at least one cell surface receptor ligand, and wherein the cell surface receptor ligand is unevenly distributed over the surface of each of the alginate-coated mesenchymal stem cells or distributed in predefined locations on the surface of each of the alginate-coated mesenchymal stem cells thereby promoting osteogenesis in the subject.

13. The method of claim 12, wherein the at least one cell surface receptor ligand is a polysaccharide and/or peptide.

14. The method of claim 12, further comprising administering an effective amount of one or more ion channel modulators, cell contractility modulators, small GTPase modulators, or a combination thereof.

15. A method of preparing a crosslinked alginate hydrogel layer encapsulating single cells, wherein the crosslinked alginate hydrogel is conjugated to at least one cell surface receptor ligand, and wherein the cell surface receptor ligand is unevenly distributed over the surface of each of the alginate-coated cells or distributed in predefined locations on the surface of each of the alginate-coated cells, comprising
(i) providing a first aqueous phase comprising alginate conjugated to a first amount of a first cell surface receptor ligand;
(ii) providing a second aqueous phase comprising unmodified alginate, cells, and a crosslinking agent;
(iii) providing a third aqueous phase comprising unmodified alginate and alginate conjugated to a second amount of the first cell surface receptor ligand or alginate conjugated to a second cell surface receptor ligand; and
(iv) simultaneously interfacing the first aqueous phase, second aqueous phase, and third aqueous phase with an oil phase that activates the crosslinking agent, thereby forming droplets comprising the crosslinked alginate hydrogel layer encapsulating single cells in which the first cell surface receptor ligand and optionally surface receptor ligand is unevenly distributed over the surface of each of the alginate-coated cells or distributed in predefined locations on the surface of each of the alginate-coated cells.

16. The method of claim 15, wherein the second aqueous phase comprises unmodified alginate and cell-CaCO₃, cell-SrCO₃, cell-BaCO₃ nanoparticles and the oil phase comprises oil and acetic acid, wherein the acetic acid releases Ca²⁺ or Sr²⁺ or Ba²⁺ from the cell-CaCO₃, cell-SrCO₃, cell-BaCO₃ nanoparticles and wherein the Ca²⁺ or Sr²⁺ or Ba²⁺ crosslinks the alginate in the first aqueous phase, second aqueous phase, and third aqueous phase to form the crosslinked alginate hydrogel layer coating single cells in which the first cell surface receptor ligand and optionally second cell surface receptor ligand is unevenly distributed over the surface of each of the alginate-coated cells or distributed in predefined locations on the surface of each of the alginate-coated cells.

17. The method of claim 15, wherein the crosslinked alginate hydrogel layer encapsulating single cells is prepared in a microfluidic device, the microfluidic device comprising three microfluidic channels for the first aqueous phase, second aqueous phase, and third aqueous phase, the three microfluidic channels converging on a fourth microfluidic channel for interfacing the first aqueous phase, second aqueous phase, and third aqueous phase with the oil phase; and an outlet channel for conveying the droplets comprising the alginate-coated cells from the fourth microfluidic channel.

18. A microfluidic device for preparing alginate-coated cells comprising
a first microfluidic channel for conveying a first aqueous phase comprising alginate conjugated to a first amount of a first cell surface receptor ligand;
a second microfluidic channel for conveying a second aqueous phase comprising unmodified alginate and cells coated with a crosslinking agent;
a third microfluidic channel for conveying a third aqueous phase comprising unmodified alginate and alginate conjugated to a second amount of the first cell surface receptor ligand or alginate conjugated to a second cell surface receptor ligand; and
a fourth microfluidic channel for conveying an oil phase comprising an oil and an agent for activating the crosslinking agent;
wherein the first, second, third and fourth microfluid channels interface at a junction whereby droplets comprising single cells encapsulated in a crosslinked alginate hydrogel layer are formed and exit the device via an outlet channel in fluid communication with the junction.

19. The microfluidic device of claim 18, wherein the first and third microfluidic channels comprise a first flow rate and the second microfluidic channel comprises a second flow rate, wherein the first flow rate and second flow rate are different.
